(19) United States
(12) Patent Application Publication
(10) Pub. No.: US 2025/0101006 A1
(43) Pub. Date: Mar. 27, 2025

(54) PROCESS FOR THE SYNTHESIS OF PYRAZOLYL DERIVATIVES USEFUL AS ANTI-CANCER AGENTS

(71) Applicant: NOVARTIS AG, Basel (CH)

(72) Inventors: Markus BAENZIGER, Basel (CH); Fabrice GALLOU, Basel (CH); Fengfeng GUO, Suzhou, Jiangsu (CN); Rudolf HÄNGGI, Basel (CH); Enjian HAN, Jinan, Shandong (CN); Guido JORDINE, Basel (CH); Jialiang LI, Suzhou, Jiangsu (CN); Weipeng LIU, Suzhou, Jiangsu (CN); Bukeyan MIAO, Suzhou, Jiangsu (CN); Shaofeng RONG, Suzhou, Jiangsu (CN); Ernesto SANTANDREA, Zürich (CH); Paul Bernd SCHIRNER, Basel (CH); Xiaodong SHEN, Suzhou, Jiangsu (CN); Can WANG, Suzhou, Jiangsu (CN); Hao ZHANG, Suzhou, Jiangsu (CN)

(21) Appl. No.: 18/834,099

(22) PCT Filed: Jan. 30, 2023
(86) PCT No.: PCT/CN2023/073833

§ 371 Date: Jul. 29, 2024

(30) Foreign Application Priority Data
Jan. 31, 2022 (WO) PCT/CN2022/075275

(51) Int. Cl. C07D 403/14; B01J 31/22; B01J 31/24

(52) U.S. Cl. CPC C07D 403/14; B01J 31/2208; B01J 31/2409; B01J 2231/4205; B01J 2531/004; B01J 2531/0263; B01J 2531/824

(57) ABSTRACT

The invention provides a novel process, novel process steps and novel intermediates useful in the synthesis of pharmaceutically active compounds, especially KRAS G12C inhibitors. The present invention provides a direct enantioselective chemical manufacturing method of making Compound A, or a pharmaceutically acceptable hydrate or solvent thereof: (I). The invention provides a process for preparing Intermediate B6* comprising reacting Intermediate B4* with Intermediate B5* in an atroposelective coupling reaction, using a chiral catalyst.

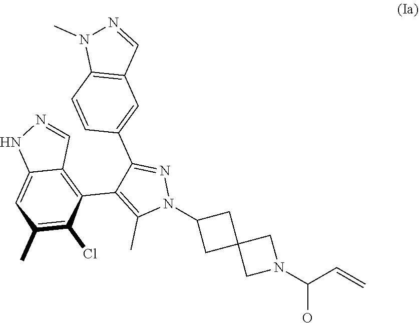

### FIELD OF THE INVENTION

The present invention relates to pyrazolyl compounds and methods for preparing these compounds. These pyrazolyl compounds are more specifically compounds which are described in WO2021/124222A1 and are useful in the treatment of cancer, and in particular KRAS G12C mutant cancer. The present invention therefore provides processes, process steps and intermediates useful in the preparation of pyrazolyl compounds such as Compound A which are useful in the treatment of cancer, and in particular KRAS G12C mutant cancer. The processes of the invention are suitable for the preparation on a large scale, e.g 500 g, 1 kg or more of the compounds described herein.

### INTRODUCTION

RAS are small GTPases acting as molecular ON/OFF switches which adopt an active/inactive state when bound to GTP/GDP, respectively. In response to growth factors, guanine exchange factors exchange GDP for GTP, turning Ras ON. RAS bound to GTP adopts conformations that recruit effector proteins to the plasma membrane, thereby activating signaling cascades causing cell growth, proliferation and survival. These cancer promoting signals are very transient and tightly controlled. They are turned off immediately by the GTPase activity of RAS itself, mainly due to the 100000 fold acceleration by GTPase activating proteins (GAPs) (Bos J L et al., Cell, Volume 129, Issue 5, 1 Jun. 2007, pp 865-877). In contrast, RAS mutants are insensitive to these GAPs, causing the RAS mutants to reside longer in the GTP bound state and shifting the GTP/GDP cycle in accordance to their intrinsic hydrolysis rate towards the ON state.

The three RAS genes constitute the most frequently mutated gene family in cancer, with RAS mutations found in ˜25% of human tumors. Among the 3 paralogs, KRAS mutations are most frequent (85% of all RAS-driven cancers), whereas NRAS and HRAS mutations are less frequently reported (12% and 3%, respectively). The majority of KRAS mutations occurs at the hotspot residues G12, G13 and Q61. KRAS G12C mutations represent about 12% of all KRAS mutations and are prevalent in lung cancer patients (˜13% lung adenoma carcinoma (LUAC)), ˜3-5% colon adenocarcinomas, a smaller fractions of other cancer types and in about 20% of MYH polyposis colorectal adenomas (COSMIC v80 database; A. Aime' et al, Cancer genet. 2015, 208:390-5).

Several orally bioavailable small-molecule KRAS G12C covalent inhibitors have entered clinical development, including sotorasib (AMG 510) and adagrasib (MRTX-849). Both also show antitumor activity against KRAS G12C-mutated tumors in early-phase clinical trials leading to the accelerated approval of sotorasib by the US FDA in May 2021 and conditional marketing authorization by the European Commission in January 2022. Nonetheless, there remains an ongoing need for improved clinical outcomes, especially since emerging data indicate multiple mechanisms of acquired resistance in patients treated with sotorasib or adagrasib.

WO2021/124222 A1 discloses compounds useful in inhibiting the G12C mutant KRAS, HRAS or NRAS proteins by forming an irreversible covalent bond with the cysteine at the position 12. Compound A is highly potent, selective covalent oral inhibitor of KRASG12C and is described in Example 1a of WO2021/124222A1. Compound A is the compound of formula (Ia):

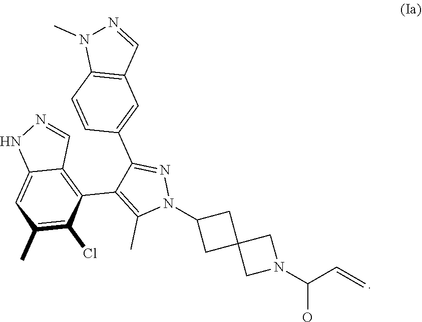

The chemical name of Compound A is “(Ra)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one”. Alternatively, the chemical name of Compound A may be written as “a(R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one”. Compound A is also known as “JDQ443” or “NVP-JDQ443”, or “1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one”.

Compound A is in clinical development as monotherapy and in combination with TNO155, with both strategies showing antitumor activity in patients with KRAS G12C-mutated tumors.

WO2021/124222 A1 is incorporated herein in its entirety, and especially for processes and intermediates useful in the preparation of Compound A and its analogues. WO2021/124222 A1 describes a synthesis of Compound A as follows:

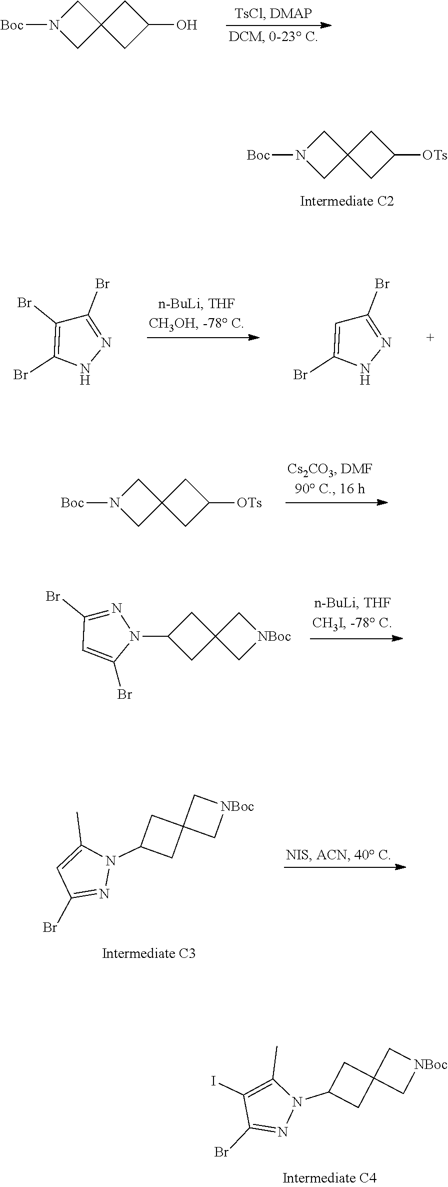

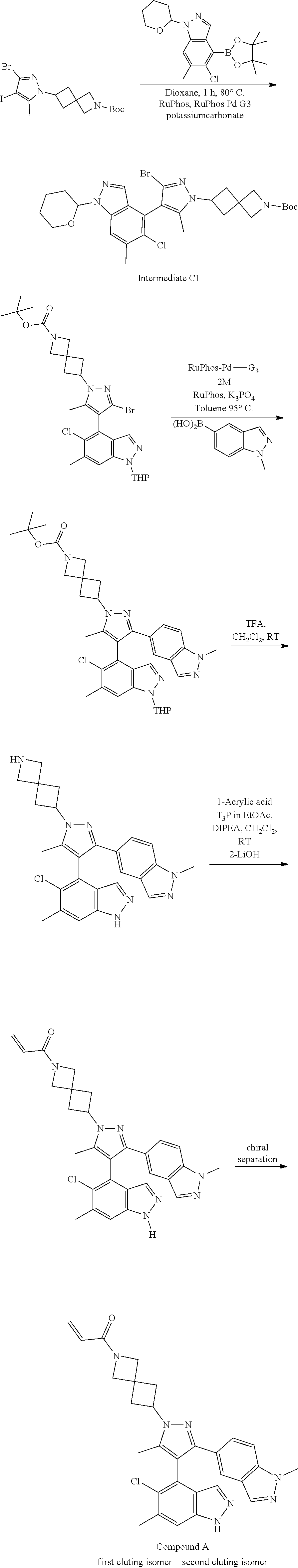

It can thus be seen that the prior art requires separation of atropisomers by chiral chromatography at the very end of the synthesis. This leads to a much-diminished yield of the desired atropisomer (only about half of the product at the end of a multi-step synthesis route is the atropisomer of interest). In addition, the synthesis disclosed in the prior art requires having to use special columns for chiral separation and high volumes of solvents used for elution. Chiral separation leads to high costs and solvent usage and significant waste. The prior art processes are thus not environmentally sustainable and require a long cycle time.

### DESCRIPTION OF THE INVENTION

The present invention provides a more efficient and convenient process for preparing Compound A. The process can be carried out on a large industrial scale and produces Compound A with the required atropisomer purity and in good yield.

In particular, the present inventors have found a process which enables the synthesis of Compound A in high enantiomeric excess by introducing atroposelectivity at a much earlier stage in a multi-step synthesis of Compound A.

Thus, useful intermediates can now be produced with good yields and atroposelectivities and used to form the end product.

In an important aspect of the invention, the present invention provides a process for preparing Intermediate B6* comprising coupling Intermediate B4* with Intermediate B5* using a chiral catalyst, wherein Intermediate B4*, B5* and B6* are as described herein.

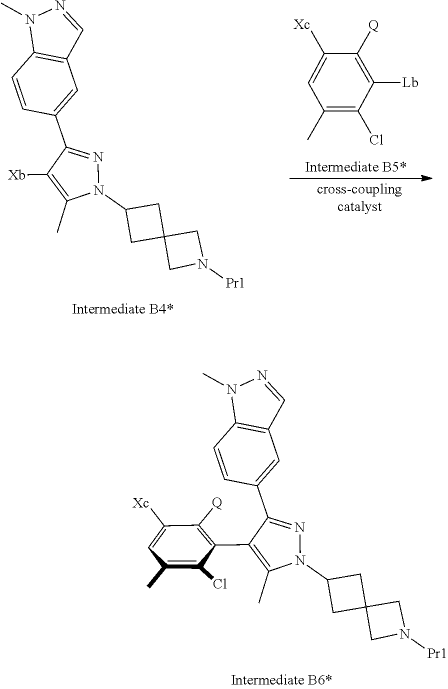

The above process may be a palladium-catalyzed atroposelective coupling of Intermediate B4* with Intermediate B5*.

According to the invention, a compound of formula (Ia) may therefore be prepared according to the synthetic scheme below.

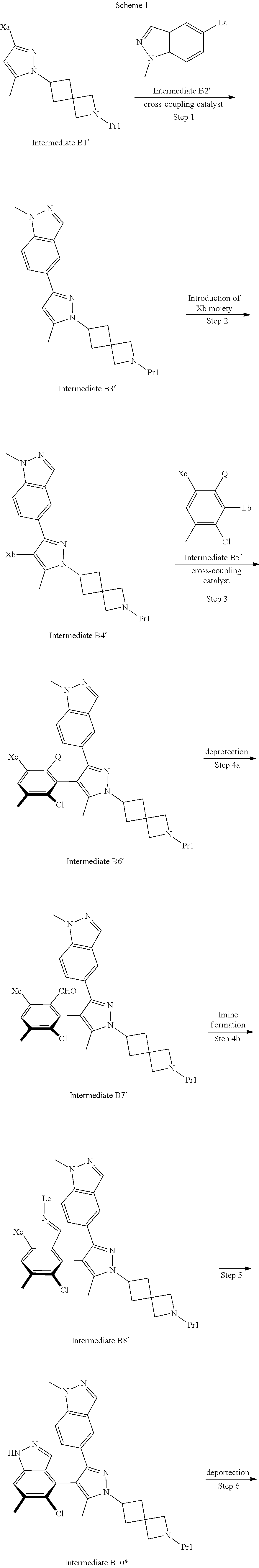

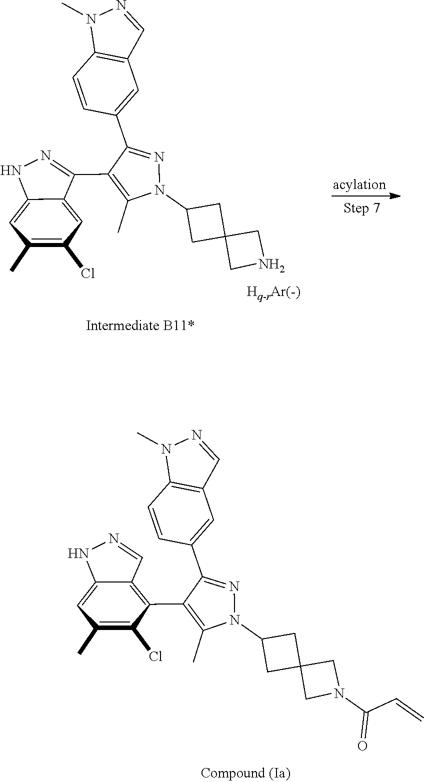

The present invention thus provides a process for preparing a compound of formula (Ia) comprising one or more of Step 1, Step 2, Step 3, Step 4a, Step 4b, Step 5, Step 6 and Step 7, and combinations thereof, wherein Step 1 to Step 7 are as described in Scheme 1.

In addition the present invention provides a process for preparing a compound which is selected from Intermediate B1*, Intermediate B2*, Intermediate B3*, Intermediate B4*, Intermediate B5*, Intermediate B6*, Intermediate B7*, Intermediate B8*, Intermediate B9*, Intermediate B10* and Intermediate B11*, as defined herein and as described in Scheme 1.

The present invention also provides a compound which is selected from Intermediate B1*, Intermediate B2*, Intermediate B3*, Intermediate B4*, Intermediate B5*, Intermediate B6*, Intermediate B7*, Intermediate B8*, Intermediate B9*, Intermediate B10* and Intermediate B11*, as described in this specification.

The present invention also provides the use of a compound which is selected from Intermediate B1*, Intermediate B2*, Intermediate B3*, Intermediate B4*, Intermediate B5*, Intermediate B6*, Intermediate B7*, Intermediate B8*, Intermediate B9*, Intermediate B10* and Intermediate B11*, as defined herein and as described in Scheme 1, for use in a process for preparing a compound of formula (Ia), or a salt, or a hydrate or a solvate thereof.

A first embodiment of the present invention is a process for preparing a compound of formula (Ia), or a salt, hydrate or solvate thereof,

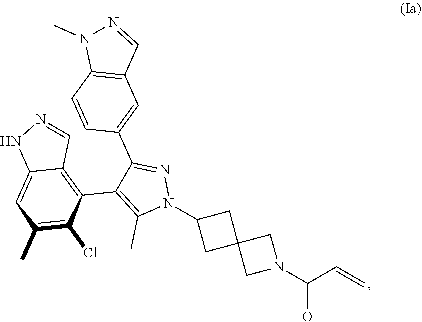

comprising reacting a compound of formula B11b*, or a salt thereof,

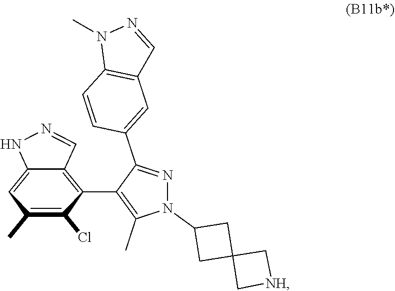

with acrylic acid or a reactive derivative of acrylic acid to form a compound of formula (I) and optionally subsequently forming a salt, hydrate or solvate of the compound of formula (Ia).

A second embodiment (also named Step A in the following) of the method comprises an acylation of Compound B11*,

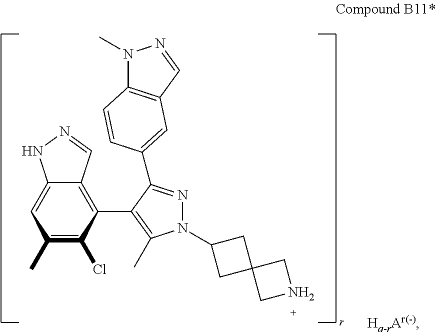

wherein r is 1, 2, 3, 4, or 5, especially 1 to 3, e.g. 1 or 2, q is 1, 2, 3, 4 or 5, especially 1 to 3, e.g. 1 or 2, and A is an acid anion of an organic or an inorganic acid, with acrylic acid or a reactive derivative thereof, or the deprotonated free form thereof without the acid anion (obtainable e.g. by treatment with a base such as an alkaline metal hydroxide, e.g. sodium hydroxide, or ammonia, at a basic pH, e.g. pH 14), to yield a compound of formula (Ia) in free form or as a hydrate or solvate.

Compound B11* is, according to another embodiment of the invention, prepared in a process (also named Step B in the following) comprising deprotecting a compound named Intermediate B10*,

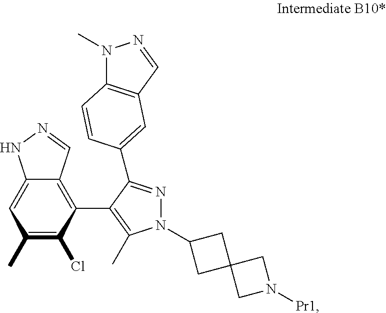

wherein Pr1 is a nitrogen-protecting group.

Removal of the protecting group in Compound B11* may be carried out under acidic conditions, i.e. in the presence of an acid. For example the acid may be an acid H_{q}A, wherein H is a hydrogen that can dissociate forming a proton, q is 1, 2, 3, 4 or 5, especially 1 to 3, e.g. 1 or 2, and A is a radical that can form an acid anion, that is, an acidic radical.

Intermediate B10* is, in another embodiment of the invention, prepared in a process (also named Step C in the following) comprising reacting hydrazine, or a hydrate or solvate thereof, with an Intermediate B8*,

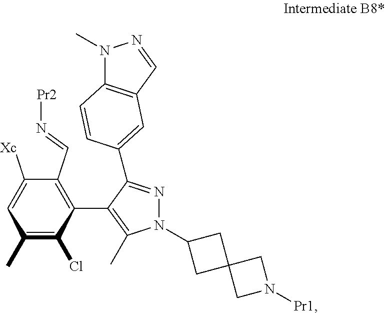

wherein Pr1 is a nitrogen-protecting group, Pr2 is a protected hydroxyl group or an unsubstituted or substituted amino group, and Xc is halogeno or pseudohalogeno.

Intermediate B8* is, in another embodiment of the invention, prepared by a process (also named Step D in the following) comprising reacting an Intermediate B7*,

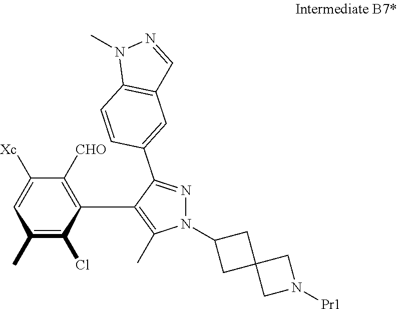

wherein Pr1 is a nitrogen-protecting group and Xc is halogeno or pseudohalogeno, with a hydroxyolamine derivative of the formula Pr2-NH₂, wherein Pr2 is a protected hydroxyl group or an unsubstituted or substituted amino group.

Intermediate B7* is, in another embodiment of the invention, prepared in a process (also named Step E in the following) comprising reacting an Intermediate B6*,

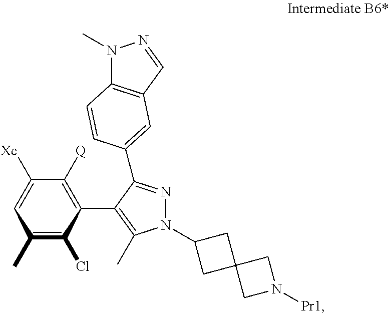

wherein Q is formyl or in particular a formyl group in the form of an acetal or (further) of a Schiff base, Pr1 is a nitrogen-protecting group and Xc is halogeno or pseudohalogeno, in the presence of an acid converting the formyl acetal group Q into free formyl.

Intermediate B6* is, in another embodiment of the invention, prepared in a process (also named Step F in the following) comprising reacting an Intermediate B4*,

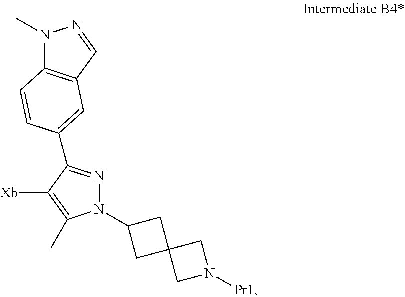

wherein Pr1 is a nitrogen-protecting group and
Xb is (a) (in particular) hydrogen, halogeno, —OSO₂RA in which RA is C₁-C₄-alkyl, fluoro-C₁-C₆-alkyl or substituted phenyl, or ORB wherein RB is C₁-C₄-alkyl, if Lb is borono (—B(OH)₂) (preferred), a boronic diester moiety (preferred), BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li;
or (b) (further) Xb is borono (—B(OH)₂) (preferred), a boronic diester moiety (preferred), BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li; if Lb is hydrogen, halogeno, —OSO₂RA in which RA is C₁-C₄-alkyl, fluoro-C₁-C₆-alkyl or substituted phenyl, or ORB wherein RB is C₁-C₄-alkyl,
where in each of (a) and (b) the moieties Xb and Lb do not have identical meanings, preferably one (especially Lb) is borono or especially a boronic diester moiety, the other (especially Xc) is —OSO₂RA in which RA is C₁-C₄-alkyl, fluoro-C₁-C₆-alkyl or substituted phenyl, ORB wherein RB is C₁-C₄-alkyl, or especially halogeno, especially iodo,
with an Intermediate B5* in an atroposelective coupling,

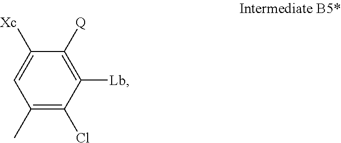

wherein Q is formyl or in particular a formyl group in the form of an acetal or a Schiff base, Lb is (a′) (in particular) borono (—B(OH)₂), a boronic diester moiety, BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li, if Xb is as just defined above for Xb under (a),
or (b′) (further) Lb is hydrogen, halogeno, —OSO₂RA in which RA is C₁-C₄-alkyl, fluoro-C₁-C₆-alkyl or substituted phenyl, or ORB wherein RB is C₁-C₄-alkyl, if Xb is as just defined under (b) for Xb above;
and Xc is halogeno or pseudohalogeno;
with the proviso that Lb and Xc are not identical, preferably one of them, especially Lb, is borono or especially a boronic ester moiety, while the other, especially Xc, is —OSO₂RA in which RA is C₁-C₄-alkyl, fluoro-C₁-C₆-alkyl or substituted phenyl, ORB wherein RB is C₁-C₄-alkyl or especially halogeno, preferably fluoro.

The atroposelective coupling may be carried out in the presence of palladium catalyst with chiral additives, or in the presence of a chiral (enantiomerically pure) mono- or (especially) bisphosphine ligand catalyst and a palladium source reagent. The palladium source reagent is in some embodiments the palladium catalyst. This step is very important as it serves to stereospecifically introduce the required atropisomery as basis for all subsequent steps leading to a compound of formula (Ia).

Intermediate B4* is, in another embodiment of the invention, if Xb is not hydrogen, prepared in a process (also named Step G in the following) comprising reacting an Intermediate B3*

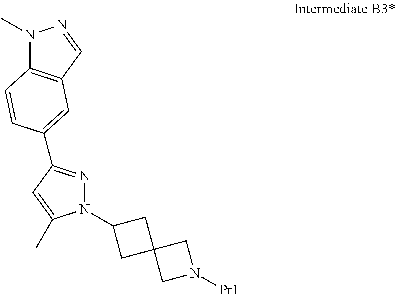

wherein Pr1 is a nitrogen-protecting group, with a reagent capable of inserting the group Xb as defined above except for hydrogen for Intermediate B4*.

Intermediate B3* (which is also an Intermediate B4* wherein Xb is hydrogen and Pr1 is a nitrogen-protecting group, so that for the synthesis thereof the preceding Step G inserting a group Xb other than hydrogen can be omitted) is, as another invention embodiment, obtained in a process (also named Step H in the following) comprising coupling Intermediate B2*,

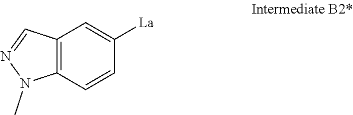

wherein
La is (A) (in particular) borono, a boronic diester moiety, BF₃K, MgX, in which X is Cl, Br or I, Zn(RZ), in which RZ is halogen, alkyl or aryl, Sn(RD)(RE)(RF), in which each of RD, RE, RF is methyl, n-butyl, Si(RG)(RX)(RY), in which each of RG, RX, RIY is hydrogen, fluoro, hydroxy, alkyl, alkoxy, or Li, if Xa is hydrogen, halogeno, —OSO₂RA in which RA is C₁-C₄-alkyl, fluoro-C₁-C₆-alkyl or substituted phenyl, or ORB wherein RB is C₁-C₄-alkyl; or
La is (B) hydrogen, halogeno, —OSO₂RA in which RA C₁-C₄-alkyl, fluoro-C₁-C₆-alkyl or substituted phenyl, or ORB wherein RB is C₁-C₄-alkyl, if Xb is borono, a boronic diester moiety, BF₃K, MgX, in which X is Cl, Br or I, Zn(RZ), in which RZ is halogen, alkyl or aryl, Sn(RD)(RE)(RF), in which each of RD, RE, RF is methyl, n-butyl, Si(RG)(RX)(RY), in which each of RG, RX, RIY is hydrogen, fluoro, hydroxy, alkyl, alkoxy, or Li, in the presence of a cross-coupling catalyst with Intermediate B1*,

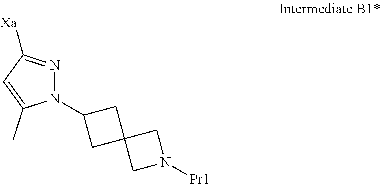

wherein Pr1 is a nitrogen-protecting group and
Xa is (A′) (in particular) hydrogen, halogeno, —OSO₂RA in which RA is C₁-C₄-alkyl, fluoro-C₁-C₆-alkyl or substituted phenyl, or ORB wherein RB is C₁-C₄-alkyl if La is as just defined for La under (A),
or Xa is (B′) borono, a boronic diester moiety, BF₃K, MgX, in which X is Cl, Br or I, Zn(RZ), in which RZ is halogen, alkyl or aryl, Sn(RD)(RE)(RF), in which each of RD, RE, RF is methyl, n-butyl, Si(RG)(RX)(RY), in which each of RG, RX, RIY is hydrogen, fluoro, hydroxy, alkyl, alkoxy, or Li, if La is as just defined for La under (B),
to yield Intermediate B3*.

The invention, in another embodiment, also provides a process for the manufacture of a compound of formula (Ia), or a hydrate or solvate thereof, comprising first Step B, then Step A, each as mentioned above or described specifically below.

The invention, in another embodiment, also provides a process for the manufacture of a compound of formula (Ia), or a hydrate or solvate thereof, comprising first Step C, then Step B and then Step A, each as mentioned above or described specifically below.

The invention, in another embodiment, also provides a process for the manufacture of a compound of formula (Ia), or a hydrate or solvate thereof, comprising first Step D, then Step C, then Step B and then Step A, each as mentioned above or described specifically below.

The invention, in another embodiment, also provides a process for the manufacture of a compound of formula (Ia), or a hydrate or solvate thereof, comprising coupling Step E, then Step D, then Step C, then Step B and then Step A, each as mentioned above or described specifically below.

The invention, in another embodiment, also provides a process for the manufacture of a compound of formula (Ia), or a hydrate or solvate thereof, comprising first Step F, then coupling Step E, then Step D, then Step C, then Step B and then Step A, each as mentioned above or described specifically below.

The invention, in another embodiment, also provides a process for the manufacture of a compound of formula (Ia), or a hydrate or solvate thereof, comprising coupling Step H, then Step G, then Step F, then coupling Step E, then Step D, then Step C, then Step B and then Step A, each as mentioned above or described below.

The invention, in another embodiment, also provides a process for the manufacture of a compound of formula (Ia), or a hydrate or solvate thereof, comprising coupling Step H, then Step G, then Step F, then coupling Step E, then Step D, then Step C, then Step B and then Step A, each as mentioned above or described below.

The invention, in another embodiment, also provides a process for the manufacture of Intermediate B8* as defined above or below, comprising Step E, then Step D, and then Step C, each as mentioned above or described specifically below.

The invention, in another embodiment, also provides a process for the manufacture of Intermediate B10* as defined above or below, comprising Step F, then Step E, then Step D and then Step C, each as mentioned above or described specifically below.

The invention, in another embodiment, also provides a process for the manufacture of a compound of the formula B11* as defined above or below, comprising Step F, then Step E, then Step D, then Step C, and then Step B, each as mentioned above or described specifically below.

Another embodiment of the invention relates to Intermediate B6* as defined above or specifically below, especially Intermediate B6.

Another embodiment of the invention relates to Intermediate B7* as defined above or specifically below, especially Intermediate B7.

Another embodiment of the invention relates to Intermediate B8* as defined above or specifically below, especially Intermediate B8.

Another embodiment of the invention relates to Intermediate B10* as defined above or specifically below, especially Intermediate B10.

Another embodiment of the invention relates to Intermediate B11* as defined above or below, especially Intermediate B111.

A special embodiment of the invention relates to a manufacture of a compound of formula (Ia), or a solvate or hydrate thereof, comprising the following reaction scheme:

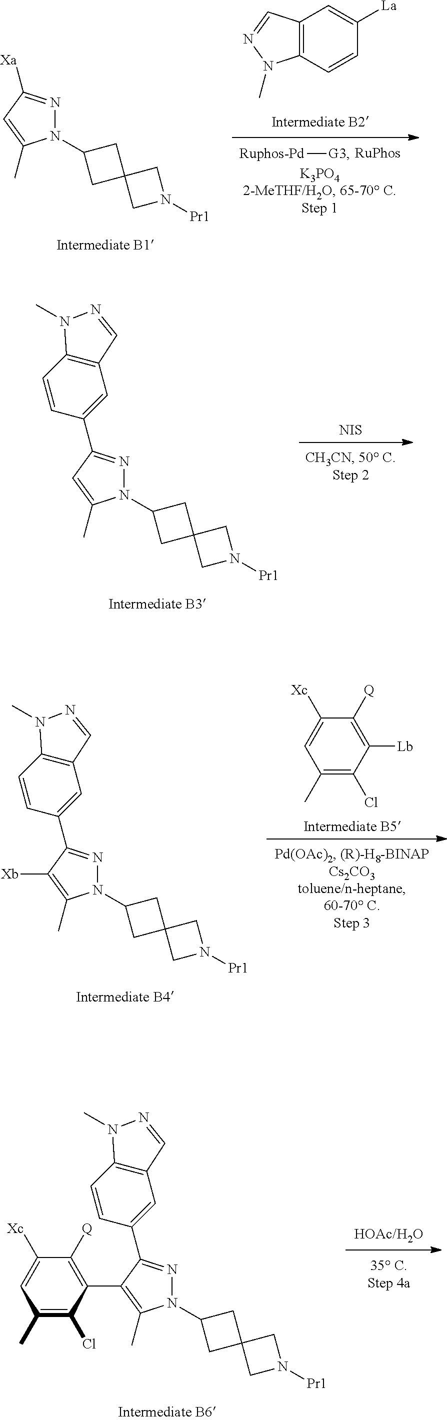

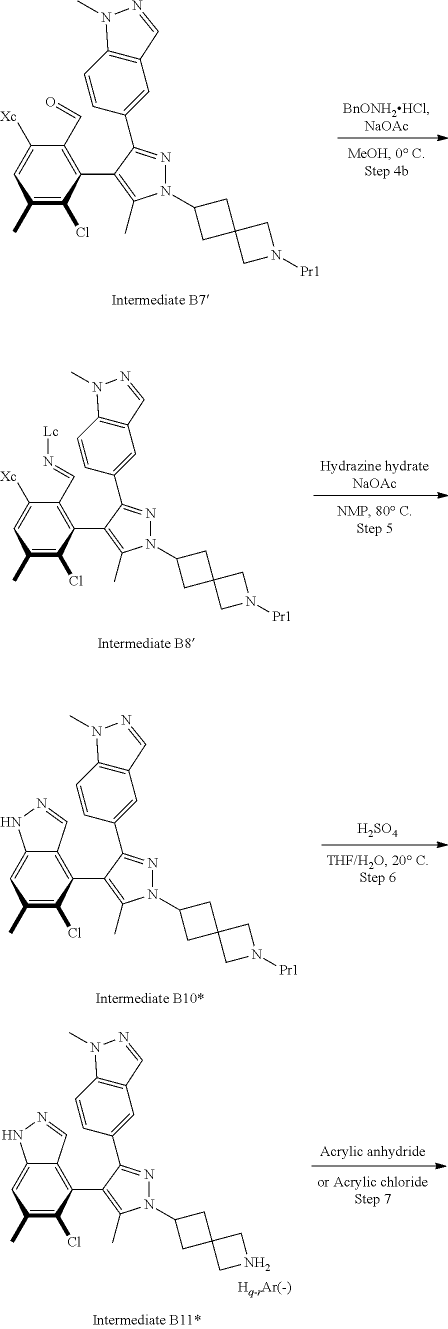

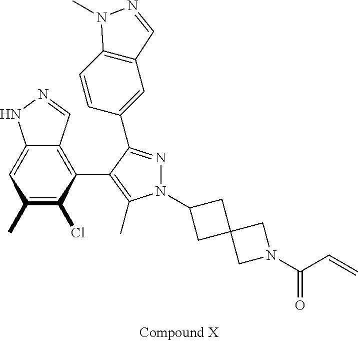

wherein for Intermediate B1′, Xa and Pr1 are as defined above or below for Intermediate B1*; for Intermediate B2′, La is as defined above or below for Intermediate B2*; for Intermediate B3′, Pr1 is as defined above for Intermediate B3*; for Intermediate B4′, Xb and Pr1 are as defined above or below for Intermediate B4*; for Intermediate B5′, Q, Xc and Lb are as defined above or below for Intermediate B5*; for Intermediate B6′, Q, Pr1 and Xc are as defined above or below for Intermediate B6*; for Intermediate B7′, Pr1 and Xc are as defined above or below for Intermediate B7*; for Intermediate B8′, Pr1, Xc and Pr2 are as defined above or below for Intermediate B8*; for Intermediate B10′, Pr1 is as defined above or below for Intermediate B10*; and for Intermediate B11′, A, q and r are as defined above for Intermediate B11*. Preferably all moieties are as defined below for the compounds B1 to B11 in the “Special Scheme” given in the Examples section.

### DETAILED DESCRIPTION OF THE INVENTION

The invention relates to any embodiment as defined above, below or in the claims.

The following definitions also apply unless otherwise provided or apparent from context and may, in each embodiment, replace one or more or all general definitions, thus leading to additional invention embodiments that are thus to be regarded as specifically disclosed herein:
a compound of formula (Ia) is an atropisomer (Eliel, E. and Wilen, S. (1994) Stereochemistry of Organic Compounds, John Wiley & Sons, Inc., pp. 1142-55).
   The enantiomeric purity is usually represented by the enantiomeric excess (“e.e.” or “ee”) which is, represented as percent (%), defined herein by the following formula:

$$ee ( \% ) = ( \vert {F}_{R} - {F}_{S} \vert \times 100 ) \%$$

wherein F_{R} is the molar fraction of the desired enantiomer and F_{S} is the molar fraction of the other enantiomer.

As used herein, the term “halogeno”, “halogen” or “halo” refers to fluoro, bromo, chloro or iodo. Halogen-substituted groups and moieties, such as alkyl substituted with halogen (halo-alkyl or halogeno-alkyl), can be mono-, poly- or per-halogenated. Chloro, or especially bromo or iodo, are especially preferred where these moieties are to be replaced in a coupling reaction.

As used herein, the term “pseudohalogeno” or “pseudohalogen” refers to is used to refer to strongly bound, linear or planar univalent radicals which can form anions, hydracids, neutral dipseudohalogens and interpseudohalogens. Examples are univalent radicals “Y” (e.g., Y═CN, OCN, N₃) that are capable of forming anions “Y—”, hydracids HY, dipseudohalogens Y₂, and interpseudohalogens, XY (X=halogen or pseudohalogen)

The substituents and features represented by variables in the Intermediates B11* to B1* as mentioned above (and also defined in more detail below) preferably have the following meanings:

A in an acid H_{q}A and in an acid anion H_{q-r}A^{r(-)} is preferably an acid group that together with the proton, in the case of an acid H_{q}A, if deprotonated forms an acid anion group. An acid H_{q}A may be an inorganic acid such as hydrochloric acid, hydrobromic acid, hydroiodic acid, sulfuric acid, sulfinic acid, pyrosulfuric acid, phosphoric acid, pyrophosphoric acid, further acidic resins, and the like. H_{q}A may be an inorganic acid such as acetic acid, propionic acid, glycolic acid, oxalic acid, acrylic acid, maleic acid, malonic acid, succinic acid, fumaric acid, tartaric acid, citric acid, benzoic acid, mandelic acid, or sulfonic acids, such as alkylsulfonic acids, e.g. methanesulfonic acid, ethanesulfonic acid, aryl sulfonic acids, such as phenylsulfonic acid, toluenesulfonic acid, sulfosalicylic acid, trifluoroacetic acid, trifluoroethanesulfonic acid, or the like. H_{q}A can also be an acid selected from the group consisting of sulphuric acid, nitric acid, phosphoric acid, acetic acid and trifluoroacetic acid.

If r protons are removed from the acid H_{q}A, the result is an anion with q-r hydrogens and r negative charges. “

The number “q” is an integer 1, 2, 3, 4 or 5, especially 1 to 3, e.g. 1 or 2.

The number “r” is an integer 1, 2, 3, 4 or 5, especially 1 to 3, e.g. 1 or 2.

Q is formyl or a formyl group in acetal or Schiff base form selected from the group consisting of
(a)

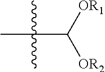

in which R₁ and R₂ are independently selected from alkyl or arylalkyl or together form an alkenyl bridge that may be unsubstituted or substituted with one or more moieties selected from alkyl, aryl and arylalkyl; some preferred moities are

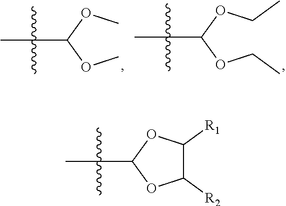

in which R₁ and R₂ are independently selected from hydrogen, alkyl and aryl,

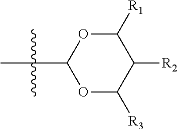

in which R₁ and R₂ are independently selected from hydrogen, alkyl and aryl; the following groups are special examples:

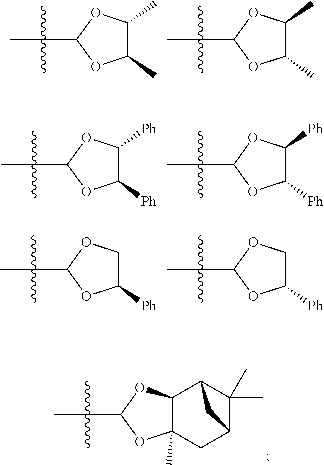

and
(b) —CH═N—R′ in which R′ is selected from the group consisting of alkyl, aryl, alkoxy and —S(═O)—R″ wherein R″ is selected from alkyl, aryl and alkoxy, such as —S(═O)-alkyl (e.g. tert-butyl) or —S(═O)phenyl.

In case the group at the position of Q is formyl (—CHO), the corresponding compound is Intermediate B7*.

Pr1 is nitrogen-protecting group (meaning a nitrogen protecting group protecting a secondary amino moiety), especially one that can be removed by not too harsh acidic conditions maintaining the integrity of the rest of the molecule to be deprotected. As used herein, the term “nitrogen-protecting group” (PG) in a compound described herein refers to a group that should protect the functional group concerned against unwanted secondary reactions, such as acylations, etherifications, esterifications, oxidations, solvolysis and similar reactions. It may be removed under deprotection conditions. Depending on the protecting group employed, the skilled person would know how to remove the protecting group to obtain the free amino moiety by reference to known procedures. These include reference to organic chemistry textbooks and literature procedures such as J. F. W. McOmie, “Protective Groups in Organic Chemistry” (1973), Springer; “Methoden der organischen Chemie” (Methods of organic chemistry), Houben-Weyl, 4th edition, Volume 15/I, Georg Thieme Verlag, Stuttgart 1974; or P. G. M. Wuts, “Greene's Protective Groups in Organic Synthesis” (e.g. 5^{th} ed., 2014), and in “Methoden der organischen Chemie” (Methods of Organic Chemistry).

Preferred nitrogen-protecting groups include: C₁-C₆alkyl (e.g. tert-butyl), preferably C₁-C₄alkyl, more preferably C₁-C₂alkyl, most preferably C₁-alkyl which is mono-, di- or tri-substituted by trialkylsilyl-C₁-C₇alkoxy (eg. trimethylsilyethoxy);
aryl, preferably phenyl, or a heterocyclic group (e.g., benzyl, cumyl, benzhydryl, pyrrolidinyl, trityl, pyrrolidinylmethyl, 1-methyl-1,1-dimethylbenzyl, (phenyl)methylbenzene) wherein the aryl ring or the heterocyclic group is unsubstituted or substituted by one or more, e.g. two or three, residues, e.g. selected from the group consisting of C₁-C₇alkyl, hydroxy, C₁-C₇alkoxy (e.g. para-methoxy benzyl (PMB)), C₂-C₈-alkanoyl-oxy, halogen, nitro, cyano, and CF₃;
arylalkyl, e.g. benzyl or methoxybenzyl (MPM=PMB)
aryl-C₁-C₂-alkoxycarbonyl (preferably phenyl-C₁-C₂-alkoxycarbonyl (eg. benzyloxycarbonyl (Cbz); benzyloxymethyl (BOM);
pivaloyloxymethyl (POM)), C₁-C₁₀-alkenyloxycarbonyl, C₁-C₆alkylcarbonyl (eg. acetyl or pivaloyl), C₆-C₁₀-arylcarbonyl; C₁-C₆-alkoxycarbonyl (eg. tertbutoxycarbonyl (Boc), methylcarbonyl, trichloroethoxycarbonyl (Troc), pivaloyl (Piv), allyloxycarbonyl), C₆-C₁₀-arylC₁-C₆-alkoxycarbonyl (e.g. 9-fluorenylmethyloxycarbonyl (Fmoc)), allyl or cinnamyl, sulfonyl or sulfenyl, succinimidyl group, silyl groups (e.g. triarylsilyl, trialkylsilyl, triethylsilyl (TES), trimethylsilylethoxymethyl (SEM), trimethylsilyl (TMS), triisopropylsilyl or tertbutyldimethylsilyl).

In embodiments of the invention, the nitrogen-protecting group is C₁-C₆-alkoxycarbonyl (eg. tertbutoxycarbonyl (Boc), methyloxycarbonyl, trichloroethoxycarbonyl (Troc), pivaloyl (Piv), allyloxycarbonyl). More preferably the nitrogen-protecting group is tert-butoxycarbonyl.

Especially preferred Pr1 is selected from the group consisting of tert-butoxycarbonyl (Boc), carbobenzoxycarbonyl (Cbz), benzyl (Bn), methoxybenzyl (MPM), trifluoroacetyl, acetyl, fluoren-9-yl-methoxycarbonyl (Fmoc) and trityl (Tr).

More preferably the nitrogen-protecting group is tert.-butoxycarbonyl (Boc).

Functional groups in any of the intermediates may optionally be protected (e.g. free NH groups in Intermediates B10*, B10′ and B10 and in Intermediates B11*, B11′ and B11) and the protecting groups may be removed at any stage as desired, so that the final compound of formula (Ia) is obtained in free, hydrate or solvate form.

Xc is halogeno or pseudohalogeno, especially selected from the group consisting of F, Cl, Br, I; CN and NO₂.

Pr2 is preferably selected from
(a) Alkyloxy and
(b) unsubstituted or substituted amino, especially NR′″R″″, in which R′″ and R″″ are independently selected from the group consisting of hydrogen and alkyl.

Xb is in case (a) mentioned above under Intermediate B4* selected from the group consisting of halogeno, especially fluoro or more especially chloro, bromo or iodo, and —OSO₂RA in which RA is C₁-C₄-alkyl, e.g. methyl (thus leading to mesylate), fluoro-C₁-C₆-alkyl, especially perfluoro-C₁-C₆-alkyl, especially as trifluoromethyl, or unsubstituted or (especially C₁-C₄-alkyl-, such as methyl-) substituted phenyl, such as in toluenesulfonyl; e.g. o-, m- or p-toluenesulfonyl; for example, Xb is a mesylate, triflate or tosylate moiety. Alternatively, it is defined as above under (b) below Intermediate B4* as borono (—B(OH)₂), a boronic diester moiety, BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li (see the particular definitions below under Lb).

Lb is (in case (a′) mentioned under formula B5* above) preferably selected from the group consisting of
(I) borono or a boronic diester moiety BR*R**, in which R* and R** are independently selected from the group consisting of alkyl and alkoxy or together form an (unsubstituted or (e.g. methyl or ethyl) substituted alkylene bridge, where the most commonly used boronate groups are selected from those with the following formulae

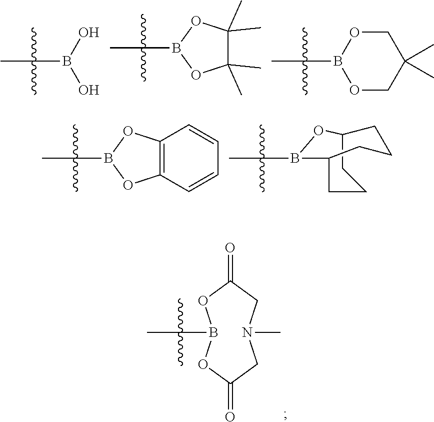

(II) BF₃K;
(III) MgX, in which X is preferably selected from Cl, Br and I;
(IV) ZnR***, in which R*** is halogen or alkyl;
(V) SnR1*R2*R3*, in which R1*, R2* and R3* are independently selected from alkyl, especially from methyl and n-butyl; and
(VI) SiR1**R2**R3**, in which R1**, R2** and R3** are independently selected from hydrogen, fluoro, hydroxyl, alkyl, especially methyl and ethyl, and alkoxy, especially methoxy or ethoxy.

Alternatively, as defined in case (b′) under formula B5* above, Lb is hydrogen, halogeno, —OSO₂RA in which RA is C₁-C₄-alkyl, fluoro-C₁-C₆-alkyl or substituted phenyl, or ORB wherein RB is C₁-C₄-alkyl as defined below for Xb; in particular, it is as defined under (a) below Intermediate B5*.

Most preferred is a borono or boronic diester moiety as defined for Lb under (I).

La is preferably, independently, selected from the moieties defined for Lb, especially of the more specific groups, such as borono or a boronic diester moiety as defined under (1) for Lb; further, it may be defined as under its definition behind (B) under Intermediate B2* above.

Lc is preferably selected from the group consisting of
(a) alkoxy, such as methoxy or ethoxy; and
(b) NR′*R*′, wherein R′* and R*′ are independently selected from hydrogen and alkyl such as methyl or ethyl.

Xa is preferably independently selected from the groups defined for Xb, in particular being defined as under (A′) above below Intermediate B1*, or as defined under (B′) above below Intermediate B1*.

Where a chiral mono- or (especially) bisphosphine ligand catalyst (and a palladium source reagent) is mentioned, this relates preferably to, as ligand, a chiral bisphosphine ligand or a monoxide thereof, e.g. (R) H₈-BINAP ([(1R)-5,5′,6,6′,7,7′,8,8′-octahydro-[1,1′-binaphthalin]-2,2′-diyl]-bis-[diphenylphosphin]), (R)—H₈-BINAPO, (R)-BINAP ((R)-2,2′-Bis-(diphenylphosphino)-1,1′-binaphthalin), (R)-BINAPO, (R)-BINAPOXyl (R)-(+)-2,2-Bis(di-3,5-Xylylphosphino)-1,1-Binaphthyl), T-BINAP ((R)-(+)-2,2′-Bis(di-p-tolylphosphino)-1,1′-binaphthyl), SEGPhos ([4(R)-(4,4′-bi-1,3-benzodioxol)-5,5′-diyl]-bis-[diphenylphosphin]), SynPhos ((R)-[5-(6-diphenylphosphanyl-2,3-dihydro-1,4-benzodioxin-5-yl)-2,3-dihydro-1,4-benzodioxin-6-yl]-diphenylphosphane), TrostPhos, MeOBIPHEP ligands (e.g. (R)-(6,6′-Dimethoxybiphenyl-2,2′-diyl)-bis[bis(3,5-di-1-tert-butyl-4-methoxyphenyl)phosphin];); or a chiral monophosphine ligand, e.g. a MOP (chiral monodentate phosphine ligand;) or a ligand ligand selected from those mentioned in WO 2011/126917 A1 (biaryl monophosphine ligand) with the following general formula Ia, Ib or a mixture thereof:

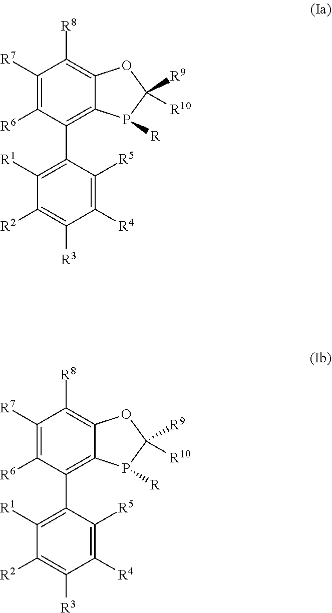

wherein:
R is (C₁-C₆)-alkyl, CF₃, (C₃-C₁₀)-carbocyclyl, 5- to 11-membered)heterocarbocyclyl, ((C₆-C₁₀)aryl, (5 to 11-membered)heteroaryl, or ferrocenyl, wherein each such (C₃-C₁₀)carbocyclyl, (5- to 11-membered)heterocarbocyclyl, (C₆-C₁₀)aryl or (5 to 11-membered)heteroaryl group is optionally substituted with 1 to 3 substituents independently selected from the group consisting of —O(C₁-C₆)alkyl, (C₁-C₆)alkyl, and CF₃;
R¹, R², R³, R⁴, R⁵ are each independently selected from the group consisting of H, halo, CF₃, —O(C₁-C₆)alkyl, (C₁-C₆)alkyl, (C₃-C₁₀)carbocyclyl, (5- to 11-membered)heterocarbocyclyl, (C₆-C₁₀)aryl, (5 to 11-membered)heteroaryl, —NR¹¹R¹², —Si(R¹¹)₃ and —SR¹¹, wherein each such (C₃-C₁₀)carbocyclyl, (5- to 11-membered)heterocarbocyclyl, (C₆-C₁₀)aryl or (5 to 11-membered)heteroaryl group is optionally substituted with 1 to 3 substituents independently selected from the group consisting of —O(C₁-C₆)alkyl, (C₁-C₆)alkyl and CF₃; or any two adjacent instances of R¹, R², R³, R⁴, R⁵, taken together with the carbons to which they are bound, form a five- or six-membered substituted or unsubstituted aryl or heteroaryl ring; provided that at least one of R¹, R², R³, R⁴, R⁵ are OR¹¹;
R⁶, R⁷, R⁸ are each independently selected from the group consisting of H, CF₃, —O(C₁-C₆)alkyl, (C₁-C₆)alkyl, (C₃-C₁₀)carbocyclyl, (5- to 11-membered)heterocarbocyclyl, ((C₆-C₁₀)aryl, (5 to 11-membered)heteroaryl and —NR¹¹R¹²; wherein each such (C₃-C₁₀)carbocyclyl, (5- to 11-membered)heterocarbocyclyl, (C₆-C₁₀)aryl or (5 to 11-membered)heteroaryl group is optionally substituted with 1 to 3 substituents independently selected from the group consisting of —O(C₁-C₆)alkyl, (C₁-C₆)alkyl, and CF₃;
R⁹, R¹⁰ are each independently selected from the group consisting of H, (C₁-C₆)alkyl, (C₃-C₆)cycloalkyl, (3- to 6-membered)heterocycloalkyl, (C₆-C₁₀)aryl, (5- to 6-membered)heteroaryl, and —SiR⁵₃; wherein each such (C₃-C₆)cycloalkyl, (3- to 6-membered)heterocycloalkyl, (C₆-C₁₀)aryl or (5- to 6-membered)heteroaryl group is optionally substituted with 1 to 3 substituents independently selected from the group consisting of —O(C₁-C₆)alkyl, (C₁-C₆)alkyl, and CF₃;
R¹¹ and R¹² are each independently selected from the group consisting of H, (C₁-C₆)alkyl, CF₃, (C₃-C₁₀)carbocyclyl, (5- to 11-membered)heterocarbocyclyl, (C₆-C₁₀)aryl, and (5 to 11-membered)heteroaryl, wherein each such (C₃-C₁₀)carbocyclyl, (5- to 11-membered)heterocarbocyclyl, (C₆-C₁₀)aryl or (5 to 11-membered)heteroaryl group is optionally independently substituted with 1 to 3 substituents independently selected from the group consisting of halo, —O(C₁-C₆)alkyl, (C₁-C₆)alkyl, and —CF₃.

The appropriate ligands in R or S form may be used as required to yield the desired result (the desired atropisomer) which can, e.g., be achieved by a simple pilot experiment, also for the other ligands mentioned below.

The ligand can, for example, be selected from the following ones:

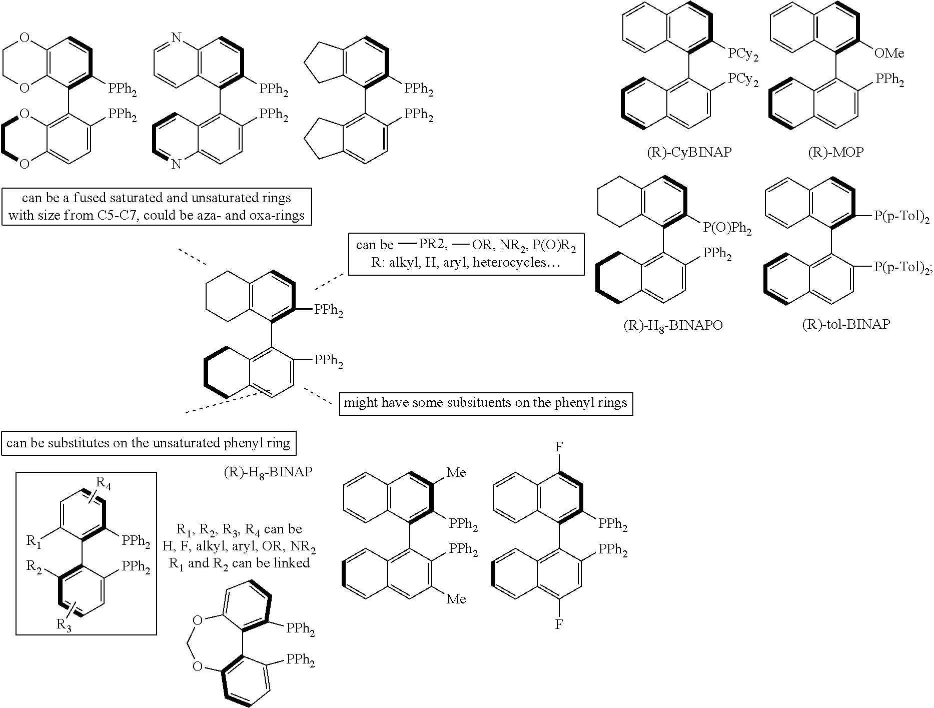

Some of the most preferred ligands are e.g. selected from the group consisting of:

### Group 1:

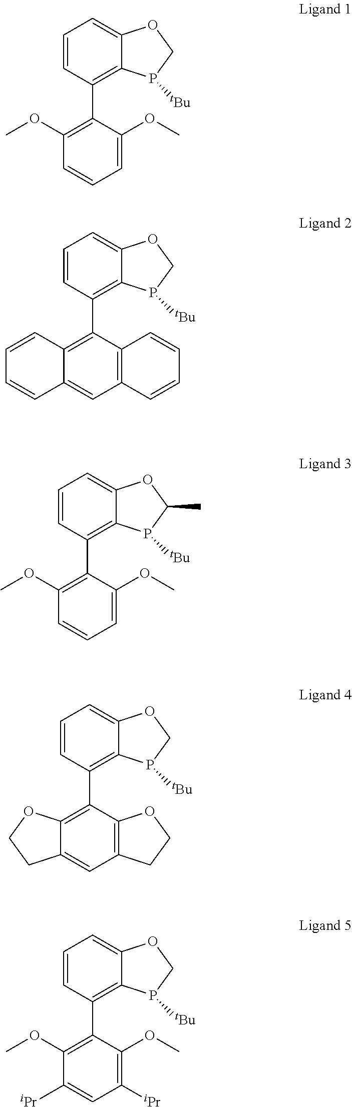

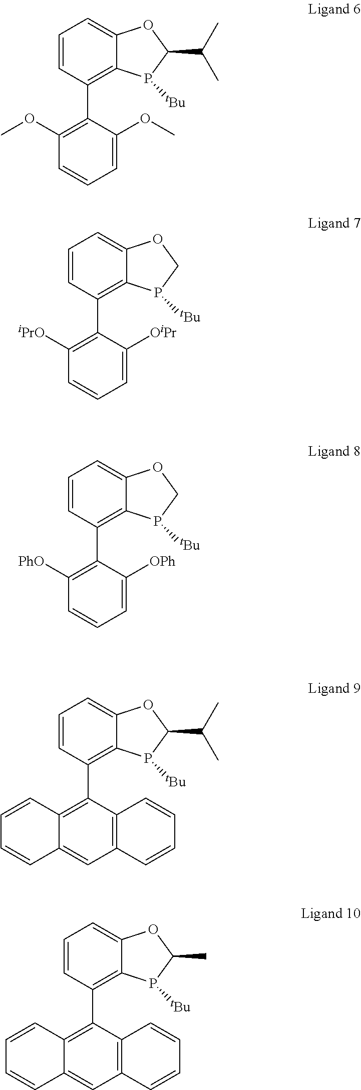 [SMILES: COc1cccc(OC)c1-c1cccc2c1[P@@](C(C)(C)C)[C@H](C(C)C)O2 ; CC(C)Oc1cccc(OC(C)C)c1-c1cccc2c1[P@@](C(C)(C)C)CO2 ; CC(C)(C)[P@@]1COc2cccc(-c3c(Oc4ccccc4)cccc3Oc3ccccc3)c21 ; CC(C)[C@@H]1Oc2cccc(-c3c4ccccc4cc4ccccc34)c2[P@@]1C(C)(C)C ; C[C@@H]1Oc2cccc(-c3c4ccccc4cc4ccccc34)c2[P@@]1C(C)(C)C]

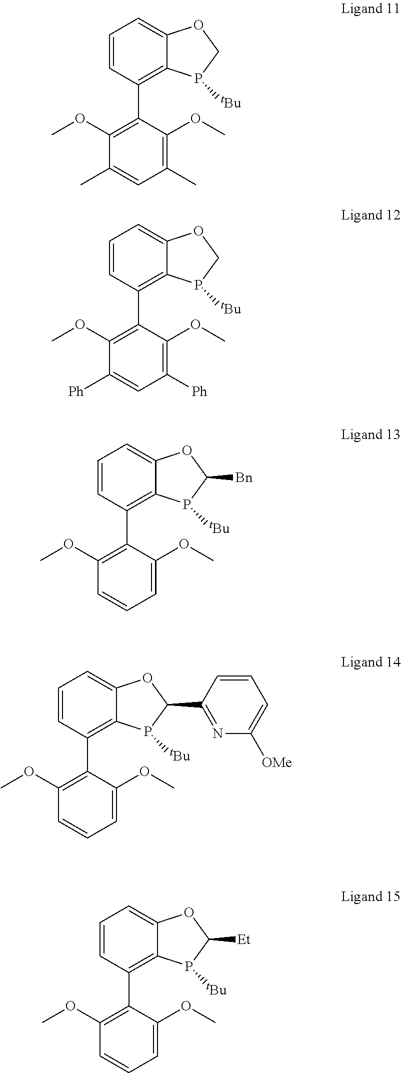

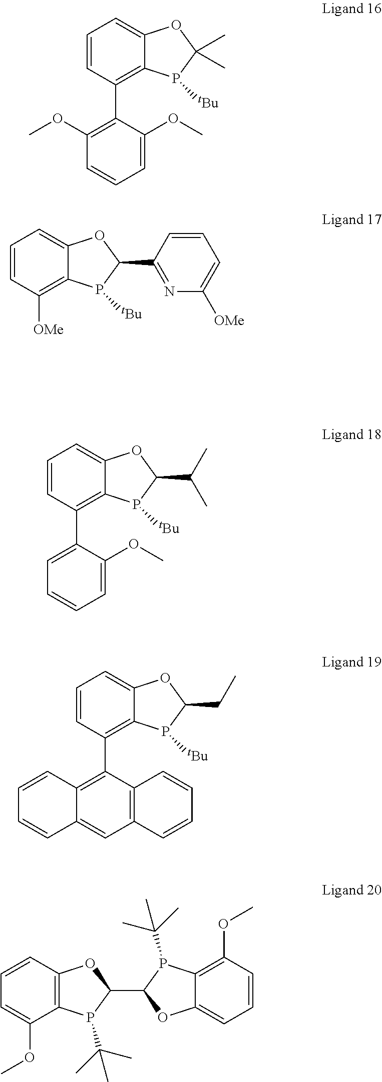

### Group 2: BINAP and Related Ligands: (Most Important are Ligand 21, 22, 23 or 24)

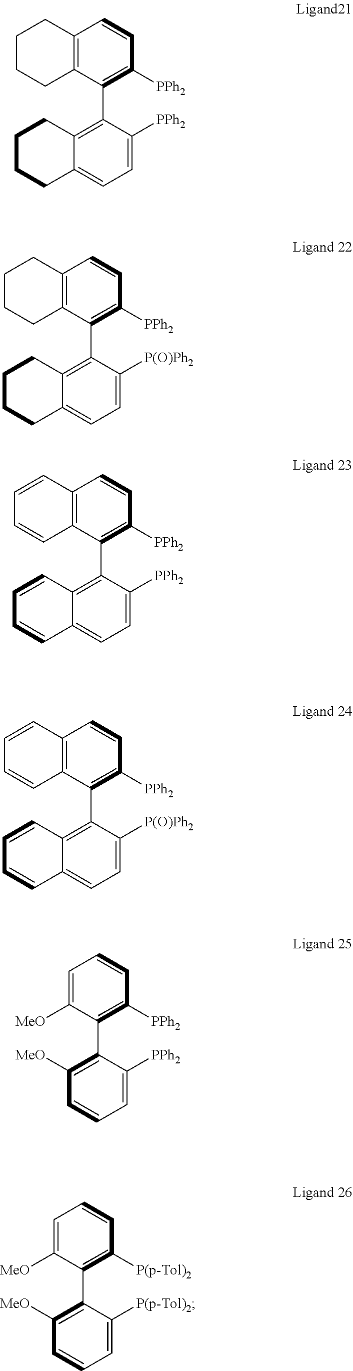

and further selected from Group 3:

### Group 3: Other Ligands:

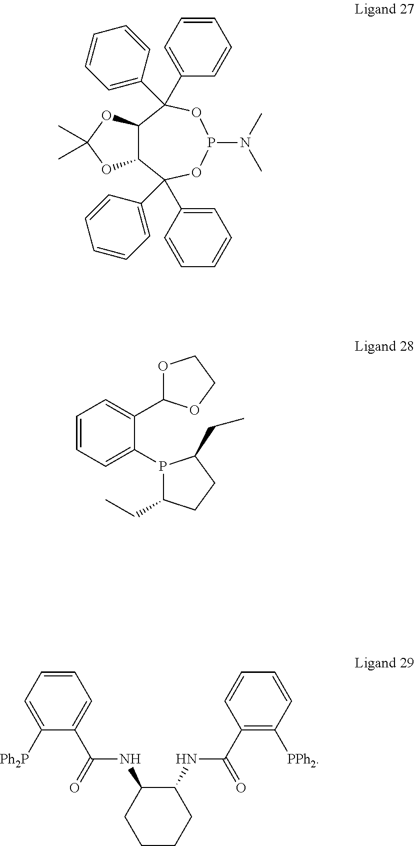

The ligands or chiral additives are available from the literature or may be purchased from vendors. For example,

| Ligand | CAS | Vendor |
|---|---|---|
| Ligand 1 | 1338454-03-7 | Strem |
| Ligand 2 | 1456816-37-7 | Strem |
| Ligand 3 | 1477517-18-2 | Strem |
| Ligand 4 | 1835717-07-1 | Strem |
| Ligand 5 | 2351219-90-2 | Daicel |
| Ligand 6 | 1477517-19-3 | Strem |
| Ligand 7 | 1338454-38-8 | Strem |
| Ligand 8 | 1441830-74-5 | Strem |
| Ligand 9 | 1891002-60-0 | Strem |
| Ligand 10 | 1477517-20-6 | Strem |
| Ligand 11 | 2351219-88-8 | Daicel |
| Ligand 12 | 2351219-89-9 | Daicel |
| Ligand 13 | 1884457-36-6 | Strem |
| Ligand 14 | n/a (catalog number in Daicel | Daicel |
| | 201126) | |
| Ligand 15 | n/a (catalog number in Strem | Strem |
| | 15-6886) | |
| Ligand 16 | n/a (catalog number in Strem | Strem |
| | 15-6882) | |
| Ligand 17 | 1542796-16-6 | Strem |
| Ligand 18 | n/a (catalog number in Daicel | Daicel |
| | 201136) | |
| Ligand 19 | n/a (catalog number in Daicel | Daicel |
| | 201141) | |
| Ligand 20 | 1228758-57-3 | Strem |
| Ligand 21 | 139139-86-9 | Strem |
| Ligand 22 | n/a | Novartis lab¹ |
| Ligand 23 | 76189-55-4 | Strem |
| Ligand 24 | 152646-80-5 | Daicel |
| Ligand 25 | 133545-16-1 | Strem |
| Ligand 26 | 133545-24-1 | Strem |
| Ligand 27 | 213843-90-4 | Strem |
| Ligand 28 | 1314246-02-0 | Sigma-Aldrich |
| Ligand 29 | 138517-61-0 | Strem |

Strem website: https://www.strem.com/index.php (e.g. Strem (Europe), Bischheim, France)
Daicel website: https://www.daicelchiraltech.cn/ (Daicel Chiral Technologies, (China) Co., Ltd) Shanghai, China),
Sigma-Aldrich website: https://www.sigmaaldrich.cn/ (Sigma-Aldrich, China)
Synthesized according to: M. Batuecas et al., ACS Catal. 2019, 9, 5268-5278.

As palladium source, any Pd salt or complex can be employed, such as Pd(OAc)₂, Pd(OPiv)₂, Pd(OCOEt)₂, PdCl₂, PdBr₂, PdI₂, Pd(OH)₂, PdSO₄, Pd(TFA)₂, Pd(dba)₂, Pd₂(dba)₃, Pd(acac)₂, Pd₂(dba)₃·CHCl₃, Pd(PPh₃)₄, Pd(PPh₃)₂Cl₂, Pd(CH₃CN)₂Cl₂, Pd(PhCN)₂Cl₂, [Pd(π-allyl)Cl]₂, [Pd(π-cinnamyl)Cl]₂, Pd[P(o-Tol)₃]₂, Pd/C, Pd(OH)₂/C, [Pd-G1]₂, [Pd-G2]₂, [Pd-G3]₂, [Pd-G4]₂, Pd(BINAP)Cl₂, Pd(dppe)Cl₂, Pd(dppp)Cl₂, Pd(dppb)Cl₂, and Pd(dppf)Cl₂; Pd(II) salts or complexes selected, e.g. for bisphosphone ligands Pd(II), for example selected from Pd(OAc)₂, Pd(TFA)₂ and Pd(PhCN)₂Cl₂; or for bisphosphine monoxide ligands Pd(0) can be used, e.g. Pd₂(dba)₃ or Pd(dba)₂.

Other appropriate Pd sources are possible and can, for example, be found in https://www.strem.com/uploads/resources/documents/buchwaldliqprecat.pdf, “Pd Metal Catalysts for Cross-Couplings and Related Reactions in the 21st Century: A Critical Review” Chemical Reviews (acs.org); or “The 2-Pyridyl Problem: Challenging Nucleophiles in Cross-Coupling Arylations”—Cook—2021—Angewandte Chemie International Edition—Wiley Online Library.

“Piv” refers to pivaloyl, “Et” to ethyl, “TFA” to trifluoroacetate, “acac” refers to acetacetonyl, “Ac” refers to acetyl, “Tol” refers to tolyl; “dppe” refers to 1,2-Bis(diphenylphosphino)ethane, “dppp” refers to 1,3-Bis(diphenylphosphino)propane, “TFA” refers to trifluoroacetyl, “dppb refers to 1,4-Bis(diphenylphosphino)butane, “dppf refers to 1,1′-Bis(diphenylphosphino)ferrocene, “Ph” to phenyl, “dba” to dibenzylideneacetone.

The reaction of Intermediate B4*, Intermediate B4′ and especially Intermediate B4 with Intermediate B5*, Intermediate B5′ or especially Intermediate B5 to Intermediate B6*, Intermediate B6′ or Intermediate B6, respectively, as defined herein above or below, preferably takes place in the presence of a base, such as selected from the group consisting of Cs₂CO₃, CsOH·H₂O, CsHCO₃, CsOPiv, CsF, CsOAc, K₃PO₄, K₂CO₃, KHCO₃, KF, TMSOK, NaOH, NaHCO₃, KOH, NaOMe, KOMe, NaOEt, KOEt, KO^{t}Bu, NaO^{t}Bu, NaOAc, KOAc, DABCO, DBU, TEA, DIPEA, Cy₂NMe, pyridine, 2,6-lutidine, or the like. Cs₂CO₃, especially in micronized form, is a preferred example. “TMSO” refers to trimethylsilanolate, “DABCO” refers to 1,4-diazabicyclo[2.2.2]octane, “DBU” refers to 1,8-diazabicyclo[5.4.0]undec-7-ene, “TEA” refers to triethylamine, “DIPEA” refers to N,N-diisopropylethylamine, “Cy” refers to cyclohexyl.

For the same reaction, which is a key reaction of the present invention and a particular embodiment, any one or more (then forming a mixture) solvents selected from the group consisting of DMF (dimethylformamide), DMSO (dimethyl sulfoxide), NMP (N-methyl-2-pyrrolidone), water, MeOH, EtOH, i-PrOH, tert-amyl alcohol, toluene, o-xylene, m-xylene, p-xylene, 1,3,5-trimethylbenzene, 1,3,5-trifluorobenzene, chlorobenzene, trifluoromethylbenzene, 1,2-difluorobenzene, n-heptane, n-hexane, c-hexane, n-pentane, THE (tetrahydrofurane), 2-MeTHF (2-methyltetrahydrofurane), 1,4-dioxane, MTBE (methyl tert-butyl ether), CPME (cyclopentyl methyl ether), i-Pr₂O, n-Bu₂O, Ph₂O, DME (1,2-dimethoxyethane), MeO(CH₂CH₂O)₂Me, cyclohexane, acetonitrile, DCM (dichloromethane), Et₃N, DIPEA (N,N-diisopropylethylamine), 2,6-lutidine, ethyl acetate, i-propyl acetate, t-butyl acetate, MIBK (methyl isobutyl ketone) and sulfolane; can, for example, be used. The sum of water in the reaction mixture preferably is kept at about 1.5 (e.g. 1 to 2, such as 1.2 to 1.8) equivalents. (on the basis of Intermediate B4*/B4′/B4), too much water or less water may lead to failure of the reaction. The reaction preferably takes place at temperatures in the range from 0° C. to the boiling temperature of the reaction mixture, especially at an elevated temperature in the range from 25 to 90° C., such as in the range from 50 to 75° C., e.g. at 60 to 70° C.

When multiple substituents are present, the substituents are selected independently unless otherwise indicated, so where 2 or 3 substituents are present, for example, those substituents may be the same or different (“independently”).

As used herein, the term “alkyl” refers to a straight or branched hydrocarbon chain radical consisting solely of carbon and hydrogen atoms, containing no unsaturation; especially to C₁-C₆-alkyl, more particularly to “C₁-C₄-alkyl”; which is attached to the rest of the molecule by a single bond. Examples of (e.g. C₁-C₄—) alkyl include, but are not limited to, methyl, ethyl, n-propyl, 1-methylethyl (iso-propyl) and n-butyl. A preferred example is methyl.

As used herein, the term alkyloxy (=alkoxy) refers to a radical of the formula —ORₐ where Rₐ is an alkyl, preferably a C₁-C₆ alkyl or especially a C₁-C₄ alkyl radical as generally defined above. Examples of C₁-C₄-alkoxy include, but are not limited to, methoxy, ethoxy, propoxy, isopropoxy and butoxy.

As used herein, the term “fluoro-alkyl” refers to an alkyl as defined herein, which is substituted with one or more fluoro or pefluoroalkyl. In particular, non-limiting examples of fluoro-C₁-C₄-alkyl include trifluoromethyl, 1,1-difluoroethyl, 2,2-difluoroethyl, 2,2,2-trifluoroethyl, 2-fluoropropyl, 3,3-difluoropropyl and 1-fluoromethyl-2-fluoroethyl. Preferred fluoro-alkyl groups, unless specified otherwise, include monofluoro-, difluoro- and trifluoro-substituted methyl and ethyl groups, e.g. CF₃, CF₂H, CFH₂, and CH₂CF₃.

As used herein, the term “aryl” refers to an aromatic hydrocarbon group having 6-14 carbon atoms in the ring portion. Typically, aryl is monocyclic, bicyclic or tricyclic aryl having 6-14 carbon atoms, often 6-10 carbon atoms, e.g., phenyl, naphthyl, fluoren-9-yl or tetrahydro-naphthyl. Phenyl is sometimes preferred. Tetrahydronaphthyl can further be included under aryl.

Where aryl is mentioned, this includes aryl carrying one or more, e.g. up to three, substituents independently selected from each other, e.g., selected from alkyl as defined before, especially methyl or ethyl, or further alkoxy as defined before, such as methoxy or ethoxy, phenyl, phenoxy, alkyl-CO—, especially acetyl or propionyl, alkyl-C(O)—O—, such as acetyloxy or propionyloxy, or yet further carboxyl (—COOH).

The term “cyano” refers to the radical —CN.

The term “amino” refers to the radical —NH₂.

The term “secondary amino” refers to a group —NH—.

The term “hydroxy” or “hydroxyl” refers to the radical —OH.

Where “further” is used in connection with a feature in an invention embodiment, it means that the features before this word are more preferred.

The term “atropisomer” refers to a stereoisomer resulting from restricted rotation about single bonds where the rotation barrier is high enough to permit isolation of the isomeric species. Typically, rotation about the single bond in the molecule is prevented, or greatly slowed, as a result of steric interactions with other parts of the molecule and the substituents at both ends of the single bond are asymmetrical, resulting in a stereogenic unit termed a “chiral axis”.

The absolute configuration of the chiral axes, for instance in exemplary compounds, is assigned using the Cahn-Ingold-Prelog (CIP) chirality rule, with stereodescriptors (aR) or (aS), or the CIP helicity rule, with stereodescriptors (P) or (M) (V. Prelog and G. Helmchen, Angewandte Chemie International Edition, 21(8): 567-583, 1982, https://doi.org/10.1002/anie.198205671; P. Mata, A. M. Lobo, C. Marshall, and A. P. Johnson, Tetrahedron: Asymmetry, 4(4): 657-688, 1993, https://doi.org/10.1016/S0957-4166(00)80173-1; both cited in H. A. Favre and W. H. Powell, Nomenclature of Organic Chemistry: IUPAC Recommendations and Preferred Names 2013 (the IUPAC “Blue Book”), Cambridge, UK: Royal Soc. of Chem., 2014, https://doi.org/10.1039/9781849733069, Chapter P-9, “Specification of Configuration and Conformation”, https://doi.org/10.1039/9781849733069-01156).

The compound of formula (Ia) can be designated by the name “(Rₐ)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one”. The compound of formula (Ia) can also be designated by the name “1-{6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one”.

The structure of Compound A is as follows.

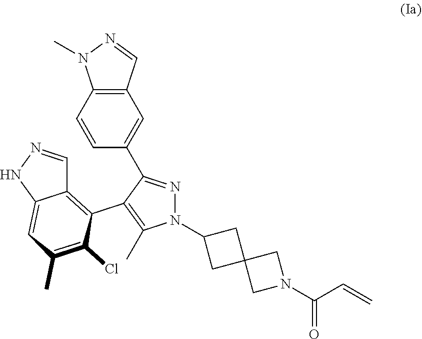

Intermediates B2*, especially B2′, more especially B2, and Intermediates B5*, especially B5′, more especially B5, are known or can be prepared according to methods known in the art or using methods as described or analogous to those described herein.

For example, Intermediate B5* may be prepared by or in analogy to the method disclosed in US 2004/44258 A1 (e.g., as described on page 165 for a compound of this Intermediate B5* type).

With regard to the intermediate B5* wherein Lb is a boronic diester moiety, among other methods readily available to the person skilled in the art, the following methods, or methods analoguous thereto, may be used for the manufacture of the Intermediate B5″ defined below:

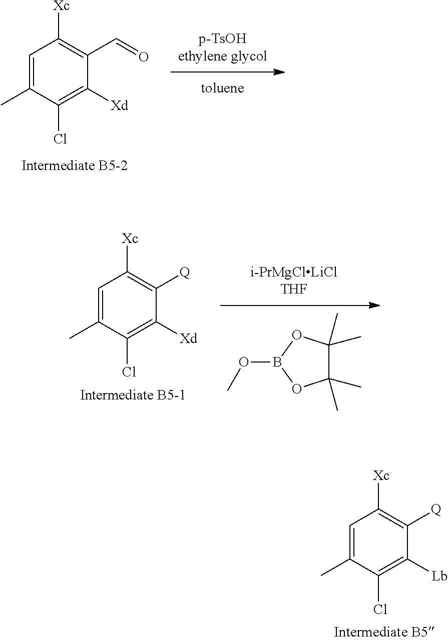

wherein Q is 1,3-dioxolanyl (or further any other formyl group in acetal form),
Xc is halogeno or pseudohalogeno, and
Xd is halogen (in particular Cl, Br or I).

Various embodiments or aspects of the invention are described herein and in particular in the claims. It will be recognized that features specified in each embodiment may be combined with other specified features to provide specific embodiments of the present invention. In particular, it will be recognized that features referred to in a particular embodiment or aspect are preferred aspects of the invention. The following enumerated embodiments are representative of the invention.

All tautomeric forms of any of the Intermediates where possible are also intended to be included. In particular, where a heteroaryl ring containing N as a ring atom is 2-pyridone, for example, tautomers where the carbonyl is depicted as a hydroxy (e.g., 2-hydroxypyridine) are included.

Where “about” is referred to in the present text, this may, for example, refer to the mentioned numeric value plus/minus 15%, such as plus/minus 10%, in particular plus/mins 5%, respectively.

As used herein, the terms “salt” or “salts” refers to an acid addition or base addition salt of an Intermediate prepared or used according to the invention. In many cases, the Intermediates are capable of forming acid and/or base salts by virtue of the presence of amino and/or carboxyl groups or groups similar thereto.

Salts can be formed with inorganic acids and organic acids. Inorganic acids from which salts can be derived include, for example, hydrochloric acid, hydrobromic acid, hydroiodic acid, sulfuric acid, sulfinic acid, pyrosulfuric acid, phosphoric acid, pyrophosphoric acid, further acidic resins, and the like. Organic acids from which salts can be derived include, for example, acetic acid, propionic acid, glycolic acid, oxalic acid, acrylic acid, maleic acid, malonic acid, succinic acid, fumaric acid, tartaric acid, citric acid, benzoic acid, mandelic acid, or sulfonic acids, such as alkylsulfonic acids, e.g. methanesulfonic acid, ethanesulfonic acid, aryl sulfonic acids, such as phenylsulfonic acid, toluenesulfonic acid, sulfosalicylic acid, trifluoroacetic acid, trifluoroethanesulfonic acid, or the like. It is to be noted that in the case of Intermediate B11*/B11′/B11, the acid forming the anion H_{q-r}A^{r(-)} may directly be acrylic acid which can then be reacted to yield a compound of formula (Ia), or a hydrate or a solvate thereof.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present invention includes Intermediates in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

For the reactions of the method embodiments mentioned above, the following conditions constitute specific embodiments:

For the reaction of Intermediate B11* to a compound of formula (Ia), the acylation takes place either with acrylic acid or preferably with a reactive derivative thereof.

The reaction with acrylic acid can, for example, take place under known conditions for the condensation of compounds with carboxylic group with compounds with an amino group, analoguous to conditions customary in peptide synthesis. For in situ formation, customary coupling agents may be applied. Such reagents are known to the person skilled in the art and can be deduced conveniently from many sources. Among the possible coupling agents for amide and ester bond synthesis the following may be mentioned: Triazoles, uronium or hexafluorophosponium derivatives, e.g. 1-hydroxy-benzotriazole (HOBt), 1-hydroxy-7-aza-benzotriazole (HOAt), ethyl 2-cyano-2-(hydroxyimino)acetate, 2-(1H-7-Azabenzotriazol-1-yl)-1,1,3,3-tetramethyluronium hexafluorophosphate methanaminium (HATU; especially preferred)), benzotriazol-1-yl-oxytripyrrolidinophosphonium hexafluorophosphate (PyBOP), 1-(mesitylene-2-sulfonyl)-3-nitro-1,2,4-triazole (MSNT), 2-(1H-benzotriazol-1-yl)-1,1,3,3-tetramethyluronium-hexafluorophosphate (HBTU), 2-(1H-benzotriazol-1-yl)-1,1,3,3-tetramethyluronium-hexafluoroborate (TBTU), 2-succinimido-1,1,3,3-tetramethyluronium-tetrafluoroborate (TSTU), 2-(5-norbornen-2,3-dicarboximido)-1,1,3,3-tetramethyluronium-tetrafluoroborate (TNTU), O-[(cyano(ethoxycarbonyl)methyliden)amino]-1,1,3,3-tetramethyluronium-tetrafluoroborate (TOTU), O-(benzotriazol-1-yl)-1,3-dimethyl-1,3-dimethylene uronium hexafluorophosphate (HBMDU), O-(benzotriazol-1-yl)-1,1,3,3-bis(tetramethylene)uronium hexafluorophosphate (HBPyU), O-(benzotriazol-1-yl)-1,1,3,3-bis(pentamethylene)uronium hexafluorophosphate (HBPipU), 3-hydroxy-4-oxo-3,4-dihydro-1,2,3-benzotriazine (HODhbt), 1-hydroxy-7-azabenzotriazole and its corresponding uronium or phosphonium salts, designated HAPyU and AOP, 1-cyano-2-ethoxy-2-oxoethylideneaminooxy-dimethylamino-morpholino-carbenium hexafluorophosphate (COMU), chlorotripyrrolidinophosphonium hexafluorophos-phate (PyCloP), or the like;

Carbodiimides, e.g. dicyclohexylcarbodiimide, N-(3-dimethylaminopropyl)-N′-ethylcarbo-diimide (=1-ethyl-3-(3-dimethyllaminopropyl)carbodiimide=EDC; especially preferred), 1-tert-butyl-3-ethylcarbodiimide, N-cyclohexyl-N′-2-morpholinoethyl)carbodiimide or diisopropylcarbodiimide (especially for ester formation via O-acyl urea formation of the carboxylic group); or
active ester forming agents, e.g. 2-mercaptobenzothiazole (2-MBT),
azide forming agents, e.g. diphenyl phosphoryl azide,
acid anhydrides, such as propane phosphonic acid anhydride,
acid halogenation agents, e.g. 1-chloro-N,N,2-trimethyl-1-propenylamine, chloro-N,N, N′,N′-bis(tetramethylene)formamidinium tetrafluoroborate or hexafluorophosphate, chloro-N,N,N′,N′-tetramethlformamidinium hexafluorophosphate, fluoro-N,N,N′,N′-tetrametylformamidinium hexafluorophosphate, fluoro-N,N,N′,N′-bis(tetramethylene)formamidinium hexafluorophosphate,
or the like, or mixtures of two or more such agents.

The reaction may, where appropriate, be conducted in the presence of a mild base (e.g. N-methylmorpholine, a trialkylamine, e.g. ethyldiisopropylamine, a di-(alkyl)aminopyridine, such as N,N-dimethylaminopyridine, or the like (taking care that the conditions are not so basic as to allow for the hydrolysis of ester groups, e.g. the depsipeptide ester group, present in precursors of the compound of the formula I), where appropriate or required in the presence of an appropriate solvent or solvent mixture, e.g. an N,N dialkylformamide, such as dimethylformamide, a halogenated hydrocarbon, e.g. dichloromethane, N-alkylpyrrolidones, such as N-methylpyrrolidone, nitriles, e.g. acetonitrile, or further an aromatic hydrocarbon, e.g. toluene, or mixtures of two or more, where, provided an excess of coupling agent is present, also water may be present. The temperatures may be ambient temperature of lower or higher, e.g. in the range from −20° C. to 50° C.

Where an active (=reactive) derivative of acrylic acid is used, the active derivative preferably is a symmetric or mixed anhydride of that acid, for example an anhydride with an inorganic acid, such as acrylic halide, especially acrylic chloride (obtainable, for example, by treatment of the acid with thionyl chloride, phosphorus pentachloride or oxalyl chloride; acid chloride method), azide (obtainable, for example, from a corresponding acid ester via the corresponding hydrazide and treatment thereof with nitrous acid; azide method), an anhydride with a carbonic acid semiderivative, such as a corresponding ester, for example carbonic acid lower alkyl semiester (obtainable, for example, by treatment of the corresponding acid with haloformic, such as chloroformic, acid lower alkyl esters or with a 1-lower alkoxycarbonyl-2-lower alkoxy-1,2-dihydroquinoline, for example 1-lower alkoxycarbonyl-2-ethoxy-1,2-dihydroquinoline; mixed O-alkylcarbonic acid anhydrides method), or an anhydride with dihalogenated, especially dichlorinated, phosphoric acid (obtainable, for example, by treatment of the corresponding acid with phosphorus oxychloride; phosphorus oxychloride method), or an anhydride with organic acids, such as a mixed anhydride with an organic carboxylic acid (obtainable, for example, by treatment of the corresponding acid with an unsubstituted or substituted lower alkane- or phenylalkane-carboxylic acid halide, for example phenylacetic acid chloride, pivalic acid chloride or trifluoroacetic acid chloride; mixed carboxylic acid anhydrides method), with an organic sulfonic acid (obtainable, for example, by treatment of a salt, such as an alkali metal salt, of the corresponding acid, with a suitable organic sulfonic acid halide, such as lower alkane- or aryl-, for example methane- or p-toluene-sulfonic acid chloride; mixed sulfonic acid anhydrides method), or with an organic phosphonic acid (obtainable, for example, by treatment of the corresponding acid with a suitable organic phosphonic anhydride or phosphonic cyanide; mixed phosphonic acid anhydrides method), and especially a symmetric anhydride (obtainable, for example, by condensation of the corresponding acid in the presence of a carbodiimide or of 1-diethylaminopropyne; symmetric anhydrides method). Preferably, an active acid derivative selected from acrylic chloride or acrylic anhydride is used. The reaction can be carried out in a manner known per se, usually in the presence of a suitable solvent or diluent or of a mixture thereof, with cooling or heating, for example in a temperature range from approximately −30° C. to approximately +150° C., especially approximately from 0° C. to +100° C., preferably from room temperature (approx. +20° C.) to +70° C., in an open or closed reaction vessel and/or in the atmosphere of an inert gas, for example nitrogen.

For the reaction of Intermediate B10* to Intermediate B11* (especially B10′ to B11′, more especially B10 to B11), which is a deprotection reaction, the protecting group Pr1 in compound B10* is preferably removed under standard conditions for the deprotection of (here secondary) nitrogen, e.g. under the conditions described below under the definition of Pr1 or in the standard textbooks and literature procedures cited there; the removal of the protecting group may typically be achieved by solvolysis (especially hydrolysis with an acid), reduction (including hydrogenation), photolysis, electrolysis or also by enzyme activity, for example under conditions analogous to physiological conditions, and that they are not present in the end-products. The person skilled in the art knows, or can easily establish, which protecting groups are suitable with the reactions mentioned hereinabove and hereinafter.

The protection of such functional groups by such protecting groups, the protecting groups themselves, and their removal reactions are described for example in standard reference books for peptide synthesis as cited hereinbefore, and in special books on protective groups such as J. F. W. McOmie, “Protective Groups in Organic Chemistry”, Plenum Press, London and New York 1973, in “Methoden der organischen Chemie” (Methods of organic chemistry), Houben-Weyl, 4th edition, Volume 15/I, Georg Thieme Verlag, Stuttgart 1974, and in T. W. Greene, “Protective Groups in Organic Synthesis”, Wiley, New York.

For example, Boc, Tr, SEM can be removed under acidic hydolysis conditions while benzyl, MPM or Cbz can be removed by hydrogenolyis, e.g. with Pd/C and hydrogen gas.

In the case of acidic conditions, the acid preferably is of the formula H_{q}A, wherein q is 1, 2, 3, 4 or 5, preferably 1, 2 or 3, and A is an acid anion, prefearbly the anion of a strong organic acid, such as trifluoroacetic acid or especially acrylic acid, or especially the anion of an inorganic acid, e.g. a monoprotonic acid, such as a halogenic acid, such as HCl or HBr, a diprotonic acid, such as sulfuric acid, or a triprotonic acid, such as phosphoric acid or the like, thus offering the advantage that the Intermediate B10* can be formed directly in its salt form of the corresponding acid. Depending on the number of protons that can be dissociated from the acid H_{q}A, the resulting anion in Intermediate 10* is of the formula H_{q-r}A^{r(-)} wherein q is as just defined, r is 1, 2, 3, 4 or 5, especially 1, 2 or 3, more especially 1 or 2.

The acidic hydrolysis can preferably be conducted at a temperature in the range from 0° C. to the boiling temperature of the reaction mixture, preferably under moderate temperature conditions to avoid decomposition of the Intermediate B10* and the resulting Intermediate B11*, for example at temperatures in the range from 5 to 30° C., preferably at a temperature around 20° C., such as at 15 to 25° C. The reaction preferably takes place in a solvent or solvent mixture, e.g. in water or an organic solvent, e.g., a cyclic ether, such as tetrahydrofurane, dioxane or the like, in dimethylformamide, in an alcohol, such as methanol, ethanol, isopropanol or ethylene glycol, or in mixtures of two or more thereof, e.g. in aqueous solutions comprising one or more of the organic solvents mentioned.

For the reaction of Intermediate B8* (especially B8′, more especially B8) with hydrazine, or a hydrate or solvate thereof, especially hydrazine or hydrazine monohydrate, to Intermediate B10*, an Intermediate B8*, as defined above or below, is reacted with hydrazine, or a hydrate or solvate thereof, under standard conditions for cyclization of a group C(H)═N-Lc (wherein Lc is as defined for Intermediate B 8*, especially B8′, more especially B8, most especially benzyloxy) with an ortho situated group Xc (as defined for Intermediate B8*, especially B8′, more especially B8; especially F). The reaction preferably takes place in the presence of an (e.g. mild or strong) base, such as an alkali or earth alkaline metal salt of an (especially up to C₆—) alkanoic acid, such as acetic acid, especially sodium acetate (but may also take place without base), in an aprotic polar organic solvent or solvent mixture, such as a cyclic ether, e.g. dioxane or tetrahydrofuran, an N,N-alkaliformamide, such as dimethyl formamide, an alcohol, such as a C₁-C₆alkanol, such as ethanol, or (especially) an N-alkyl pyrrolidone, such as N-methyl-2-pyrrolidone. The reaction preferably takes place at a temperature between 20° C. to the boiling temperature of the reaction mixture, e.g. at 50 to 90° C., such as about 80° C.

For the reaction of Intermediate B7* (especially B7′, more especially B7) to Intermediate B8*, especially B8′, more especially B8), a compound of the formula Lc-NH₂ (wherein Lc is as defined for a compound of the formula B8*) or in particular of the formula benzyl-O—NH₂, or a salt thereof, such as the salt of an organic or an inorganic acid, e.g. sulfuric acid or a hydrogen halide, such as HCl, is reacted under standard conditions for the formation of a Schiff base. For example, the reaction preferably takes place in the presence of an (e.g. mild or strong) base, such as an alkali or earth alkaline metal salt of an (especially up to C₆—) alkanoic acid, such as acetic acid (but may also take place in the absence of a base), especially sodium acetate, in a protic solvent, e.g. water in the presence of a acetic acid) or in an aprotic polar organic solvent or solvent mixture, such as a cyclic ether, e.g. dioxane or tetrahydrofuran, an N,N-alkaliformamide, such as dimethyl formamide, an N-alkyl pyrrolidone, such as N-methyl-2-pyrrolidone, or especially an alcohol, such as a C₁-C₆alkanol, such as ethanol, preferably in the presence of water. The reaction preferably takes place at a temperature between −20° C. to 30° C., e.g. at −5 to 10° C., such as at about 0° C. of the reaction mixture, e.g. at 50 to 90° C., such as about 80° C.

For the reaction of Intermediate B6* (wherein Q is preferably as defined above or below, especially being a formyl group in (preferably cyclic) acetal form) to Intermediate B7* (especially B6′ to B7′, more especially B6 to B7), customary (especially hydrolysis) conditions for the setting free of a formyl group from an acetal or Schiff base derivative can be employed, e.g. acetal cleavage in the presence of an acid, such as an organic acid, e.g. an (especially up to C₆—) alkanoic acid, such as acetic acid, in an aqueous solvent or solvent mixture. The reaction preferably takes place at a temperature in the range from 0° C. to the boiling point of the reaction mixture, e.g. in the range from 20 to 50° C., such as at about 35° C.

The reactions in the preceding paragraph are preferably conducted sequentially in a “one pot” synthesis.

The reaction of Intermediate B4*, Intermediate B4′ and especially Intermediate B4 with Intermediate B5*, Intermediate B5′ or especially Intermediate B5 to Intermediate B6*, Intermediate B6′ or especially Intermediate B6, respectively, as defined herein above or below, preferably takes place in the presence of a base, such as selected from the group consisting of Cs₂CO₃, CsOH·H₂O, CsHCO₃, CsOPiv, CsF, CsOAc, Na₂CO₃, K₃PO₄, K₂CO₃, KHCO₃, KF, TMSOK, NaOH, NaHCO₃, KOH, NaOMe, KOMe, NaOEt, KOEt, KO^{t}Bu, NaO^{t}Bu, NaOAc, KOAc, DABCO, DBU, TEA, DIPEA, Cy₂NMe, pyridine, 2,6-lutidine, or the like. Cs₂CO₃, especially in micronized form, is a preferred example. “TMSO” refers to trimethylsilanolate, “DABCO” refers to 1,4-diazabicyclo[2.2.2]octane, “DBU” refers to 1,8-diazabicyclo[5.4.0]undec-7-ene, “TEA” refers to triethylamine, “DIPEA” refers to N,N-diisopropylethylamine, “Cy” refers to cyclohexyl.

For the same reaction, which is a key reaction of the present invention and a particular embodiment, any one or more (then forming a mixture) solvents selected from the group consisting of DMF (dimethylformamide), DMSO (dimethyl sulfoxide), NMP (N-methyl-2-pyrrolidone), water, alcohols, such as MeOH, EtOH, i-PrOH or tert-amyl alcohol, aromatic solvents (preferred), such as toluene, o-xylene, m-xylene, p-xylene, 1,3,5-trimethylbenzene, 1,3,5-trifluorobenzene, chlorobenzene, trifluoromethylbenzene, 1,2-difluorobenzene or further benzene, alkanes (preferred), such as heptane, e.g. n-heptane, n-hexane, c-hexane or n-pentane, cyclic ethers, such as THF (tetrahydrofurane), 2-MeTHF (2-methyltetrahydrofurane) or 1,4-dioxane, alkyl, cycloalkyl or aryl ethers, such as MTBE (methyl tert-butyl ether), CPME (cyclopentyl methyl ether), i-Pr₂O, n-Bu₂O, Ph₂O, DME (1,2-dimethoxyethane), MeO(CH₂CH₂O)₂Me, cyclic hydrocarbons, such as cyclohexane, alkylnitriles such as acetonitrile, haloalkanes, such as DCM (dichloromethane), amines, such as Et₃N, DIPEA (N,N-diisopropylethylamine) or 2,6-lutidine, alkyl esters, such as ethyl acetate, i-propyl acetate or t-butyl acetate, ketones, such as MIBK (methyl isobutyl ketone) and sulfolane; can, for example, be used. The sum of water in the reaction mixture preferably is kept at about 1.5 (e.g. 1.2 to 1.8) equivalents (defined with respect to 1 mol of Intermediate B4*/B4′/B4 as basis), too much water or less water may lead to failure of the reaction. The reaction preferably takes place at temperatures in the range from 0° C. to the boiling temperature of the reaction mixture, especially at an elevated temperature in the range from 25 to 90° C., such as in the range from 50 to 75° C., e.g. at 60 to 70° C.

Depending on the moiety Lb under (a) to (f) above, in the case of (a) a Suzuki coupling, in the case of (b) a coupling under appropriate conditions for coupling aryl-BF₃K reagents, in the case of (c) a Kumada coupling, in the case of (d) a Negishi coupling, in the case of (e) a Stille or Hiyama coupling, and in the case of (f) a desilylative coupling may be used, for example.

For the reaction of Intermediate B3* to B4* (especially B3′ to B4′, more especially B3 to B4), a reagent capable of a substitution reaction introducing a group Xb as defined above (other than hydrogen) or below is reacted under conditions known in the art in order to introduce the group Xb. Among the reagents, halogens, such as Br₂, Cl₂ or further Cl₂; or further tetrabutylammonium bromide, pyridinium hydrobromide perbromide, 1,3-dibromo-5,5-dimethylhydantoin, 2,4,4,6-tetrabromocyclohexa-2,5-dienone, or analogues thereof; or in particular N-halogenosuccinimides (e.g. iodosuccinimide, bromosuccinimide or chlorosuccinimide) or reactive derivatives of a compound of the formula HO—SO₂RA (with RA as defined above or below) may be used, such as the acid halogenide thereof (halogeno instead of HO).

The reaction preferably takes place in an inert organic protic or aprotic solvent or solvent mixture, such as a nitrile, e.g. acetonitrile, or acetic acid. The temperature is preferably in the range from 0° C. to the boiling point of the reaction mixture, e.g. in the range from 25 to 80° C., such as about 50° C.

For example, —OTf or —OSO₂(CF₂)₃CF₃ may be inserted as follows:

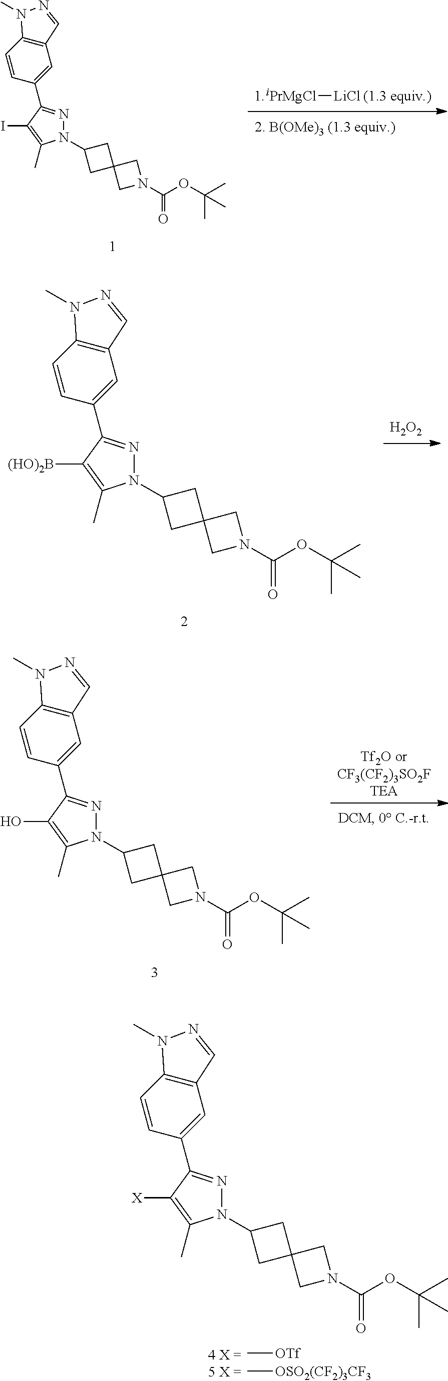

### Experiment Procedure:

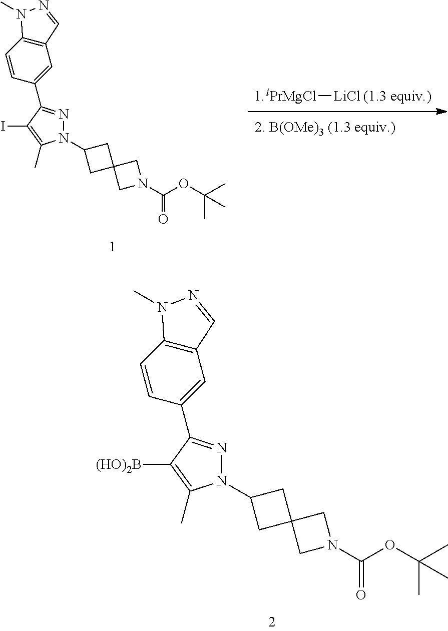

To a solution of compound 1 (2 mmol) in 6 mL of anhydrous THE was added i-PrMgCl—LiCl (2.6 mmol, 1.3 M in THF) dropwise at −10° C. under N₂ atmosphere. The resulting solution was allowed to stir under −10° C. for 60 minutes till UPLC indicate the full conversion of the starting material. B(OMe)₃ (2.6 mmol) was added in one portion at −10° C. The cold bath was removed and the resulting mixture was allowed to stir for ˜3 hours under room temperature (r.t). followed by quenching with saturated NH₄Cl (aq). The organic layers were separated, and the aqueous layer was extracted with ethyl acetate. The combined organic layer was washed with brine and dried with Na₂SO₄. The crude mixture after concentration was additionally purified by slurrying in heptane to afford the desired product.

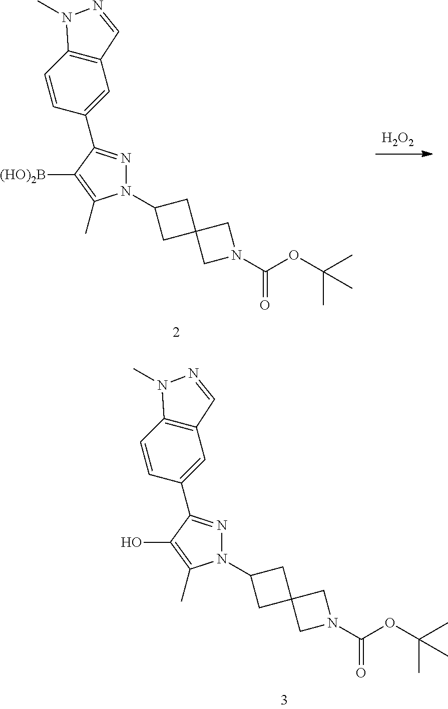

To a vigorously stirred solution of 2 (0.66 mmol) in 10 mL of THE was added NaOH (25 wt % aq.) (1.3 mmol) and H₂O₂ (30% aq.) (2.0 mmol) at 0° C. After 30 minutes, another H₂O₂ (30% aq.) (2.0 mmol) was added and the mixture was stirred at 0° C. for ˜2 h till UPLC indicate the full conversion. The resulting mixture was quenched with saturated NH₄Cl (aq), diluted with water then extracted with DCM. The combined organic layers were dried with Na₂SO₄ and concentrated under vacuo. The crude product was purified by column chromatography to afford the desired product.

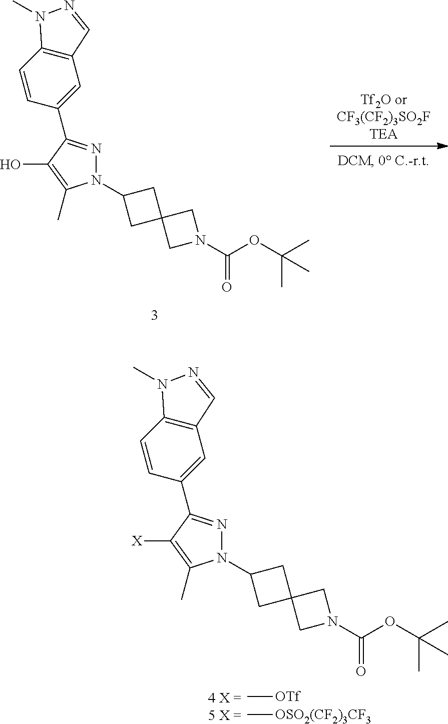

To a solution of 3 (2 mmol) and TEA (3 mmol) in 5 mL of DCM was added Tf₂O (trifluoromethanesulfonic anhydride) or nonafluoro-n-butanesulfonyl fluoride (2.4 mmol) dropwise at 0° C. The resulting suspension was stirred at room temperature till the full conversion of the starting material. The suspension was filtered and concentrated. The crude product was purified with column to afford the desired product.

For the reaction of Intermediate B1* and Intermediate B2* to Intermediate B3* (especially B1′ and B2′ to B3′, more especially B2 and B3 to B4), reaction conditions supporting the cross coupling of Intermediates B1* and B2* (especially B1′ and B2′, more especially B1 and B2) are used. For the coupling reaction of Intermediate B2* with an Intermediate B1*, in the presence of a cross-coupling catalyst (especially a Suzuki- or Stille type cross-coupling reaction, the coupling partners B2* and B1* are preferably reacted in the presence of a palladium catalyst such as RuPhos-Pd-G3/RuPhos in an appropriate solvent, such as 1,4-dioxane (or toluene) in the presence of a base (e.g. as defined above for the reaction of Intermediates B4* and B5*), especially K₃PO₄ or Na₂CO₃. Depending on the moiety La used and defined for Lb under (a) to (f) above, in the case of (a) a Suzuki coupling, in the case of (b) a coupling under appropriate conditions for coupling aryl-BF₃K reagents, in the case of (c) a Kumada coupling, in the case of (d) a Negishi coupling, in the case of (e) a Stille or Hiyama coupling, and in the case of (f) a desilylative coupling may be used, for example. See, e.g., Pd Metal Catalysts for Cross-Couplings and Related Reactions in the 21st Century: A Critical Review|Chemical Reviews (acs.org) or The 2-Pyridyl Problem: Challenging Nucleophiles in Cross-Coupling Arylations—Cook—2021—Angewandte Chemie International Edition—Wiley Online Library.

### For

the reactions wherein, in Intermediate B4*/B4′/B4, Xb and Lb are as defined under (b) above below the formula of Intermediate B4*; and
for the reactions wherein, in Intermediate B2*/B2′/B2, La and Xb are as defined under (B) above below the formula of Intermediate B2*;
   the reactions above may be made using the mutually corresponding exchanged reactions just described, respectively.

In all reactions, it is preferably possible that the resulting compounds are isolated, e.g. by standard procedures, such as solvent distribution, centrifugation or other sedimentation, precipitation such as crystallization, chromatography, filtration or the like.

The reactions, where appropriate or required, can be conducted under an inert gas, such as nitrogen, argon or helium, or carbon dioxide.

Where appropriate, reactions may be quenched, e.g. by the addition of bases, such as sodium carbonate.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁷O, ¹⁸O, ¹⁸F, ³⁵S, ³⁶C, respectively. The invention includes various isotopically labeled compounds as defined herein, for example those into which radioactive isotopes, such as ³H and ¹⁴C, or those into which non-radioactive isotopes, such as ³H and ¹⁴C are present. Such isotopically labelled compounds are useful in metabolic studies (with ¹⁴C), reaction kinetic studies (with, for example ²H and ¹³C), detection or imaging techniques, such as positron emission tomography (PET) or single-photon emission computed tomography (SPECT) including drug or substrate tissue distribution assays, or in radioactive treatment of patients. In particular, an ¹⁸F compound may be particularly desirable for PET or SPECT studies. Isotopically-labeled compounds of formula (Ia) can generally be prepared by conventional techniques known to those skilled in the art or by processes analogous to those described in the accompanying Examples using an appropriate isotopically-labeled reagents in place of the non-labeled reagent previously employed.

Alternatively, substitution with heavier isotopes, particularly deuterium (i.e., ²H or D), may afford certain therapeutic advantages resulting from greater metabolic stability, for example increased in vivo half-life or reduced dosage requirements or an improvement in therapeutic index. It is understood that deuterium in this context is regarded as a substituent of a compound of formula (Ia). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The invention also relates to the compounds of any of the embodiments mentioned wherein one or more hydrogen atoms in one or more substituents are replaced with deuterium, e.g. all hydrogens in one or more alkyl substituents are replaced with deuterium (the respective moiety/moieties are then perdeuterated).

In embodiments of the invention, alkyl (or methyl) may be deuterated or perdeuterated, in particular, when the alkyl (or methyl) is present as substituent in the Intermediates of the invention and/or when the alkyl (or methyl) is present as a substituent.

The present invention also provides the manufacture of a crystalline form of the a compound of formula (Ia), as defined herein, such as the hydrate (Modification HA) crystalline form, or the isopropyl alcohol (IPA) solvate crystalline form, or the ethanol (EtOH) solvate crystalline form or the propylene glycol solvate 30 crystalline form of a compound of formula (Ia). These and their manufacture are described in WO2021/24222 A1.

As described therein, the hydrate (Modification HA) crystalline form of a compound of formula (Ia) can be obtained, for example, from the isopropyl (IPA) solvate, ethanol (EtOH) solvate, methanol solvate, and propylene glycolate solvate of a compound of formula (Ia). The hydrate (Modification HA) crystalline form of a compound of formula (Ia) may be characterized by an X-ray powder diffraction pattern (XRPD) (measured as described in WO 2021/24222 A1) comprising at least one, two, three or four peaks having an angle of refraction 2θ values (CuKα λ=1.5418 Å) selected from the group consisting of 8.2°, 11.6°, 12.9° and 18.8°, measured at a temperature of about 25° C. and an x-ray wavelength, λ, of 1.5418 Å. The hydrate (Modification HA) crystalline form may also be characterized by an x-ray powder diffraction pattern (XRPD) comprising at least one, two, three or four or all peaks having an angle of refraction 2θ values (CuKα λ=1.5418 Å) selected from the group consisting of 8.2°, 11.6°, 12.1°, 12.9°, 14.6°, 16.2°, 18.8°, 20.4° and 24.1°, measured at a temperature of about 25° C. and an x-ray wavelength, λ, of 1.5418 Å. For example, the hydrate Modification HA can be obtained as described in Example 97 of WO 2021/24222 A1 and can also characterized in more detail as described in said document.

The isopropyl alcohol (IPA) solvate of a compound of formula (Ia) may be characterized by an X-ray powder diffraction pattern (XRPD) comprising at least one, two, or three peaks having an angle of refraction 2θ values (CuKα λ=1.5418 Å) selected from the group consisting of 7.5°, 12.5° and 17.6° measured at a temperature of about 25° C. and an x-ray wavelength, λ, of 1.5418 Å. The isopropyl alcohol solvate of a compound of formula (Ia) may be characterized by an x-ray powder diffraction pattern (XRPD) comprising at least one, two, three or four or more, or all peaks having an angle of refraction 2θ values (CuKα λ=1.5418 Å) selected from the group consisting of 7.5°, 12.5°, 15.5°, 16.4°, 17.6°, 21.4° and 24.4°, measured at a temperature of about 25° C. and an x-ray wavelength, λ, of 1.5418 Å.

The ethanol (EtOH) solvate of a compound of formula (Ia) may be characterized by an X-ray powder diffraction pattern (XRPD) comprising at least one, two, or three or four peaks having an angle of refraction 2θ values (CuKα λ=1.5418 Å) selected from the group consisting of 7.9°, 12.7°, 18.2° and 23.1°, measured at a temperature of about 25° C. and an x-ray wavelength, λ, of 1.5418 Å. The ethanol solvate of a compound of formula (Ia) may be characterized by an x-ray powder diffraction pattern (XRPD) comprising at least one, two, three or four or more, or all peaks having an angle of refraction 2θ values (CuKα λ=1.5418 Å) selected from the group consisting of 7.9°, 12.7°, 13.1°, 15.5°, 15.9°, 16.9°, 18.2°, 18.6°, and 23.1°, measured at a temperature of about 25° C. and an x-ray wavelength, λ, of 1.5418 Å.

The propylene glycol solvate of a compound of formula (Ia) may be characterized by an X-ray powder diffraction pattern (XRPD) comprising at least one, two, or three or four peaks having an angle of refraction 2θ values (CuKα λ=1.5418 Å) selected from the group consisting of 7.3°, 13.2°, 18.0° and 22.5°, measured at a temperature of about 25° C. and an x-ray wavelength, λ, of 1.5418 Å. The propylene glycol solvate of a compound of formula (Ia) may be characterized by an x-ray powder diffraction pattern (XRPD) comprising at least one, two, three or four or more, or all peaks having an angle of refraction 2θ values (CuKα λ=1.5418 Å) selected from the group consisting of 7.3°, 13.2°, 15.6°, 16.2°, 18.0°, 22.5°, 22.8°, 23.2° and 25.1°, measured at a temperature of about 25° C. and an x-ray wavelength, λ, of 1.5418 Å.

Any resulting mixtures of isomers can be separated on the basis of the physicochemical differences of the constituents, into the pure or substantially pure optical isomers, diastereoisomers, atropisomers, racemates, for example, by chromatography and/or fractional crystallization. For example, further, any one or more of the chiral Intermediates B6*/B6′/B6, B7*/B7′/B7, B8*/B8′/B8, B10*/B10′/B10 and B11*/B11′/B11 can be purified from its other enantiomer by customary methods, e.g. as described in the following paragraph:

Any resulting racemates of final products or intermediates can be resolved into the optical enantiomers by known methods, e.g., by separation of the diastereomeric salts thereof, obtained with an optically active acid or base, and liberating the optically active acidic or basic compound. In particular, a basic moiety may thus be employed to resolve the compounds of the present invention into their optical enantiomers, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic or enantiomerically impure products can also be resolved by chiral chromatography, e.g., high pressure liquid chromatography (HPLC) using a chiral adsorbent.

Traces or low amounts of an undesired enantiomer may be removed e.g. using re-crystallization using pure enantiomer (e.g. obtained by chiral chromatography) as seed material.

Typically, the Intermediates and a compound of formula (Ia) can be prepared according to the Schemes provided above and below. The more specific descriptions and the examples which outline specific synthetic routes, and the generic schemes below provide guidance to the synthetic chemist of ordinary skill in the art, who will readily appreciate that the solvent, concentration, reagent, protecting group, order of synthetic steps, time, temperature, and the like can be modified as necessary.

In another aspect, the present disclosure provides a pharmaceutical composition comprising a compound of the present invention, obtained by synthesizing a compound of formula (Ia) according to the invention, or a pharmaceutically acceptable hydrate or solvate thereof, and a pharmaceutically acceptable carrier, and especially the manufacture of such a pharmaceutical composition comprising the synthesis of a compound of formula (Ia), or a pharmaceutically acceptable hydrate or solvate thereof, and admixing it with one or more pharmaceutically acceptable carrier. In another embodiment, the composition comprises at least two pharmaceutically acceptable carriers, such as those described herein. For purposes of the present invention, unless designated otherwise, solvates and hydrates are generally also considered compositions.

Compound A (wherever mentioned, referring to the compound in free or hydrate or solvate form) is, for example, useful in the treatment of a cancer which is selected from lung cancer (such as lung adenocarcinoma and non-small cell lung cancer), colorectal cancer (including colorectal adenocarcinoma), pancreatic cancer (including pancreatic adenocarcinoma), uterine cancer (including uterine endometrial cancer) and rectal cancer (including rectal adenocarcinoma); more suitably, lung cancer, colorectal cancer or pancreatic cancer or a solid tumor, wherein the cancer is KRAS G12C-mutant. More suitably, the cancer to be treated by the compound of the invention is KRAS G12C-mutant lung cancer, including KRAS G12C-mutant non-small cell lung cancer.

The invention also relates to the embodiments mentioned in the claims, which are therefore to be regarded as included here as part of the description.

The following examples, while also defining specific invention embodiments, serve to illustrate the invention without limiting the scope thereof.

Abbreviations used are those conventional in the art.

### Abbreviations

| Abbreviation | Description |
|---|---|
| AcOH | acetic acid |
| aq | aqueous |
| BnONH₂•HCl | o-benzylhydroxylamine hydrochloride |
| DCM | dichloromethane |
| DMSO | dimethyl sulfoxide |
| DMSO-d₆ | hexadeuterodimethyl sulfoxide |
| ethyl acetate | ethyl acetate |
| e.e. | enantiomeric excess |
| ESI | electrospray ionizsation |
| G | gram(s) |
| GC | gas chromatography |
| H | hour(s) |
| IPA | Isopropanol |
| | liter/milliliter/microliter |
| Kg | kilogram |
| LC | liquid chromatography |
| MCC | microcrystalline cellulose |
| 2-MeTHF | 2-methyltetrahydrofuran |
| MeOH | Methanol |
| min | minute(s) |
| mol | molar |
| MS | mass spectroscopy |
| MTBE | methyl tert-butyl ether |
| MW, mw | microwave |
| m/z | mass to charge ratio |
| NaOAc | sodium acetate |
| NIS | N-iodosuccinimide |
| NMP | N-methylpyrrolidone |
| NMR | Nuclear Magnetic Resonance |
| PDA | photodiode array |
| i-PrMgCl LiCl | isopropylmagnesium chloride lithium chloride complex |
| | solution |
| (R)-H₈-BINAP | (R)-2,2′-bis(diphenylphosphino)-5,5′,6,6′,7,7′,8,8′- |
| | octahydro-1,1′-binaphthyl |
| RuPhos | 2-dicyclohexylphosphino-2′,6′-diisopropoxybiphenyl |
| RuPhos-Pd-G3 | (2-dicyclohexylphosphino-2′,6′-diisopropoxy-1,1′- |
| | biphenyl)[2-(2′-amino-1,1′-biphenyl)]palladium(II) |
| | methanesulfonate |
| SFC | supercritical fluid chromatography |
| THF | tetrahydrofuran |
| p-TosOH | p-toluene sulfonic acid |

### General Methods and Conditions:

Temperatures are given in degrees Celsius. If not mentioned otherwise, all evaporations are performed under reduced pressure, typically between about 15 mm Hg and 100 mm Hg (=20-133 mbar).

Mass spectra were acquired on LC-MS, SFC-MS, or GC-MS systems using electrospray, chemical and electron impact ionization methods with a range of instruments of the following configurations: Waters Acquity UPLC with Waters SQ detector or Mass spectra were acquired on LCMS systems using ESI method with a range of instruments of the following configurations: Waters Acquity LCMS with PDA detector. [M+H]⁺ refers to the protonated molecular ion of the chemical species.

NMR spectra were run with Bruker Ultrashield™ 400 (400 MHz), Bruker Ultrashield™600 (600 MHz) and Bruker Ascend™ 400 (400 MHz) spectrometers, both with and without tetramethylsilane as an internal standard. Chemical shifts (δ-values) are reported in ppm downfield from tetramethylsilane, spectra splitting pattern are designated as singlet (s), doublet (d), triplet (t), quartet (q), multiplet, unresolved or more overlapping signals (m), broad signal (br). Solvents are given in parentheses. Only signals of protons that are observed and not overlapping with solvent peaks are reported.

X-ray powder diffraction (XRPD) patterns described herein were obtained as described in WO2021/124222 A1.

The following “Scheme 2” exemplifies the steps of a synthesis according to the invention, the steps thereof also being described in the following:

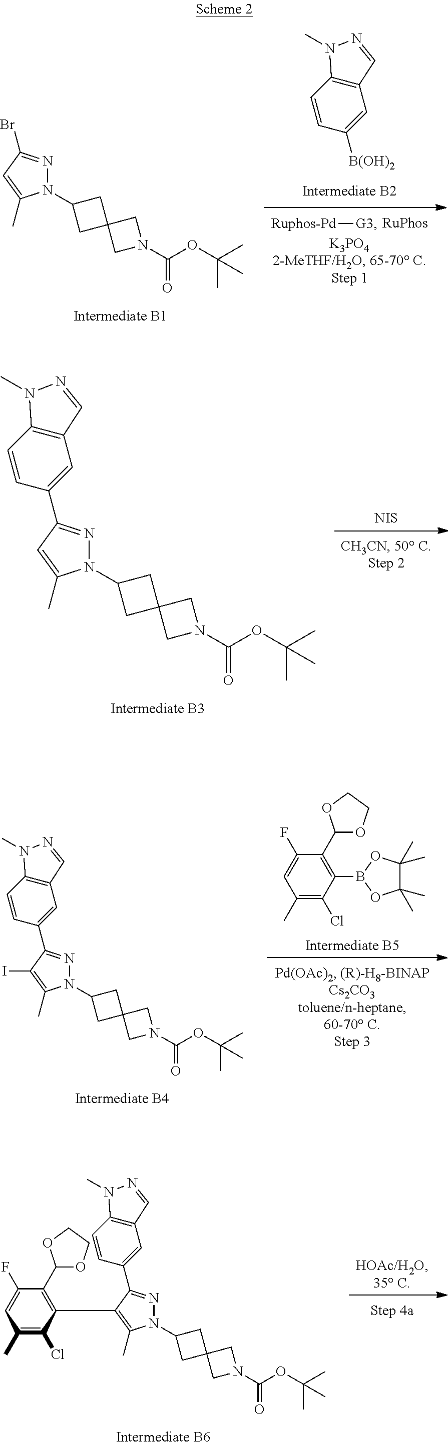

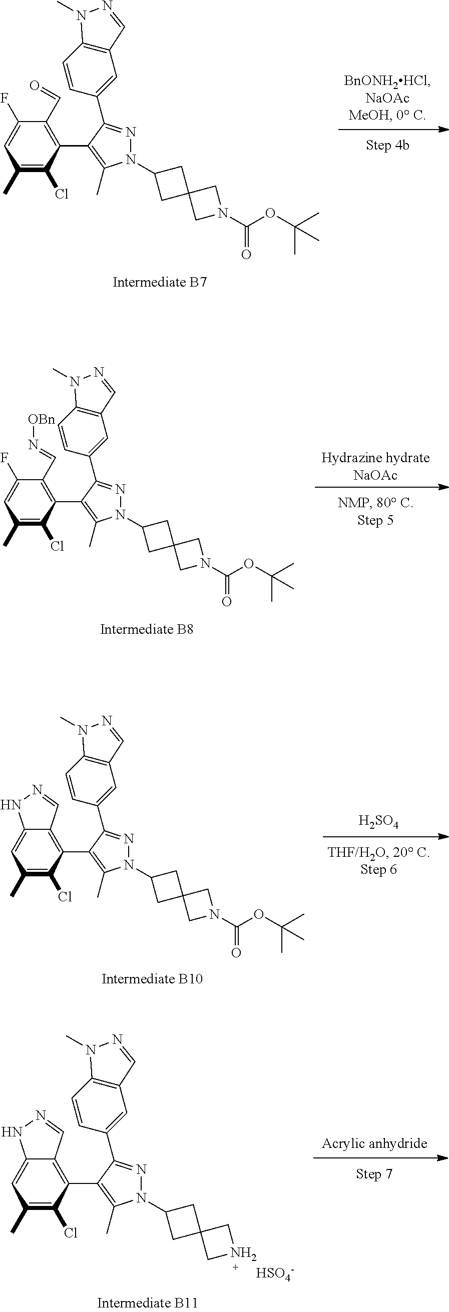

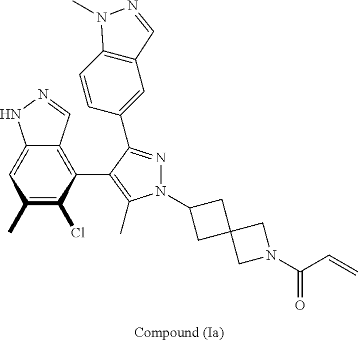

### Example 1: Synthesis of a Compound of Formula (Ia) Using Steps According to the Invention

### Step 1: tert-Butyl 6-[5-methyl-3-(1-methyl-1H-indazol-5-yl)-1H-pyrazol-1-yl]-2-azaspiro[3.3]hepta ne-2-carboxylate (Intermediate B3)

To a 500 mL three-necked flask equipped with a magnetic stirring bar, tert-butyl 6-(3-bromo-5-methyl-1H-pyrazol-1-yl)-2-azaspiro[3.3]heptane-2-carboxylate (B1, 20.00 g, 56.14 mmol, see page 192 of WO 2021/124222), (1-methyl-1H-indazol-5-yl)boronic acid (Intermediate B2, 10.08 g, 57.26 mmol, see page 165 of US 2004/44258 A) RuPhos (0.524 g, 1.12 mmol) and RuPhos-Pd-G3 (0.939 mg, 1.12 mmol) were suspended in 2-MeTHF (86.34 g) under N₂. K₃PO₄ aqueous solution (prepared by adding 17.88 g of K₃PO₄ in 100.08 g of water) was added and the resulting mixture was stirred overnight under 65-70° C. The mixture was cooled to 55-65° C. After phase separation, the organic layer was heated to 60-70° C. Active charcoal (2.00 g) was added to the organic layer and the resulting suspension was stirred under the same temperature for 4-5 h. After hot filtration, the cake was washed with 2-MeTHF twice (12.87 g×2). Water (50.00 g) was added to the combined filtrate under 60-70° C. followed by the addition of the H₃PO₄ buffer solution (2.50 g). The resulting mixture was stirred for 1 h, then the aqueous layer was removed under 60-70° C. The mixture was concentrated to ˜80 g, heated till a clear solution was obtained, then cooled to 50-60° C. The B3 crystal seeds (the seeds could be obtained from the same manufacturing batch) was added and the resulting suspension was stirred at 50-60° C. for 1 h. n-Heptane (137.19 g) was added in 4 h and the suspension was then stirred for 1 h, cooled to 25° C. in 2 h, hold overnight then filtered under vacuum. The wet cake was washed with n-heptane/2-MeTHF twice and dried under vacuum to afford the title compound as a beige powder (20.79 g, yield: 91%). The procedure has been scaled up to 300 Kg. ¹H NMR (400 MHz, DMSO-d₆) δ 8.08 (s, 1H), 8.04 (s, 1H), 7.86 (dd, J₁=8.0 Hz, J₂=1.4 Hz, 1H), 7.62 (d, J=8.0 Hz, 1H), 6.47 (s, 1H), 4.71 (p, J=8.0 Hz, 1H), 4.04 (s, 3H), 3.98 (s, 2H), 3.91 (s, 2H), 2.78-2.60 (m, 4H), 2.25 (s, 3H), 1.39 (s, 9H); ¹³C NMR (100 MHz, DMSO-d₆) δ 155.4, 149.1, 139.2, 139.1, 132.6, 126.4, 124.1, 123.7, 116.4, 109.6, 102.2, 78.4, 46.5, 39.8, 35.3, 31.3, 28.0, 10.5; MS m/z [M+H]⁺ 408.2.

Preparation of H₃PO₄ buffer solution: K₃PO₄ (14.41 g) was added to deionized water (110.4 g). 85% H₃PO₄ in H₂O (40.4 g) was added and the pH value was confirmed as 7.31.

### Step 2: tert-Butyl 6-[4-iodo-5-methyl-3-(1-methyl-1H-indazol-5-yl)-1H-pyrazol-1-yl]-2-azaspiro [3.3]heptane-2-carboxylate (Intermediate B4)

To a 350 ml four-necked round bottom flask equipped with an impeller stirrer, tert-butyl 6-[5-methyl-3-(1-methyl-1H-indazol-5-yl)-1H-pyrazol-1-yl]-2-azaspiro[3.3]heptane-2-carboxylate (B3, 20.38 g, 50.00 mmol) and acetonitrile (40.87 g) were added under N₂. The resulting suspension was heated to 50° C. followed by the addition of a NIS (12.38 g, 55.00 mmol) solution in acetonitrile (39.31 g) in 1 h. The resulting suspension was allowed to stir under 50° C. until the full conversion of B3 (approximately in 1-2 h), after which Na₂CO₃ aqueous solution (prepared by dissolving 2.65 g of Na₂CO₃ in 20.4 g of deionized water) was added dropwise in 30 min. The resulting suspension was then stirred for 15 min under 50° C. followed by the dropwise addition of deionized water (61.2 g) in 90 min and Na₂SO₃ aqueous solution (prepared by dissolving 3.15 g of Na₂SO₃ in 20.4 g of deionized water) in 30 min sequentially. The resulting suspension was stirred at 50° C. for 15 min, cooled to 25° C. in 2 h, stirred for an extra hour then filtered under vacuum. The wet cake was washed with water/acetonitrile=4/1 (v/v, 117.19 g) and dried under vacuum to afford the title compound as a white powder (25.33 g, yield: 95%). The procedure has been scaled up to 400 Kg. ¹H NMR (400 MHz, DMSO-d₆) δ 7.94 (s. 1H), 7.57 (d, J=8.0 Hz, 1H), 7.50 (d, J=8.0 Hz, 1H), 4.67 (p, J=8.0 Hz, 1H), 3.89 (s, 1H), 3.79 (s, 2H), 3.68 (s, 2H), 2.35-2.27 (m, 7H), 2.13 (s, 3H), 1.19 (s, 9H); ¹³C NMR (100 MHz, DMSO-d₆) δ 155.3, 149.7, 149.2, 139.1, 132.8, 126.3, 125.7, 123.2, 119.5, 109.3, 78.4, 61.0, 47.8, 39.6, 35.4, 31.2, 28.0, 11.6; MS m/z [M+H]⁺ 534.1.

### Step 3: tert-Butyl 6-[(4M)-4-[2-chloro-6-(1,3-dioxolan-2-yl)-5-fluoro-3-methylphenyl]-5-methyl-3-rea(1-methyl-1H-indazol-5-yl)-1H-pyrazol-1-yl]-2-azaspiro[3.3]heptane-2-carboxylate (Intermediate B6)

To a 1 L Radley reactor equipped with an impeller stirrer, tert-butyl 6-[4-iodo-5-methyl-3-(1-methyl-1H-indazol-5-yl)-1H-pyrazol-1-yl]-2-azaspiro[3.3]heptane-2-carboxylate (B4, 50.00 g, 93.74 mmol), Pd(OAc)₂ (2.10 g, 9.37 mmol), (R)—H₈-BINAP (6.50 g, 10.31 mmol), toluene (242.00 g) and n-heptane (85.50 g) were added under N₂. The resulting solution was heated to 65° C. and stirred for 30 min. Micronized Cs₂CO₃ (91.62 g, 281.21 mmol, 400-600 mesh) was added in one portion and the resulting suspension was stirred at 65° C. for 10 min. A solution of 2-[2-chloro-6-(1,3-dioxolan-2-yl)-5-fluoro-3-methylphenyl]-4,4,5,5-tetramethyl-1,3,2-dioxaborolane (B5, 48.17 g, 140.60 mmol) in toluene/n-heptane=2/1 (112.00 g) was slowly added in 26 hours via a peristaltic pump and the resulting suspension was stirred under 65° C. for another 8 h. The sum of water in the reaction mixture is kept at about 1.5 equiv., too much water or less water may lead to failure of the reaction. After the reaction finished, 9% N-acetyl cysteine aqueous solution (550.00 g, 163.19 mmol) was added under 65° C. and the resulting biphasic solution was stirred for another 7 h then cooled to 25° C. The aqueous layer was removed and the remained organic layer was filtered through MCC and washed with water (100.00 g). After phase separation, the organic layer was concentrated to around 200 g under vacuum. n-Heptane (80.00 g) and B6 crystal seeds (which ere obtained from the same manufacturing batch) was added in one-portion and the resulting suspension was stirred under 35° C. for 2 h. n-Heptane (120.00 g) was added dropwise in 2 h and the resulting suspension was stirred for 2 h, cooled to 20° C. and filtered under vacuum. The wet cake was washed with toluene/n-heptane=2/1 (99.90 g) and dried under vacuum to afford the title compound as an off-white powder (45.65 g, yield: 78%, 98.2% e.e.). The procedure has been scaled up to 200 Kg (yield: 79-81%, 99.6% e.e.). ¹H NMR (400 MHz, DMSO-d₆) δ 7.93 (s, 1H), 7.62 (s, 1H), 7.49 (d, J=8.0 Hz, 1H), 7.44-7.33 (m, 2H), 5.60 (s, 1H), 4.83 (p, J=8.0 Hz, 1H), 4.06-3.88 (m, 7H), 3.88-3.66 (m, 4H), 2.90-2.64 (m, 4H), 2.34 (s, 3H), 1.95 (s, 3H), 1.39 (s, 9H); ¹³C NMR (100 MHz, DMSO-d₆) δ 160.7, 158.2, 155.4, 147.1, 139.4 (J=9.0 Hz), 138.8, 137.7, 134.8 (J=5.0 Hz), 132.6, 130.6 (J=3.0 Hz), 126.3, 124.9, 124.4 (J=11.0 Hz), 123.4, 118.4 (J=23.0 Hz), 117.6, 112.1, 109.3, 100.1, 78.4, 65.2 (J=4.0 Hz), 47.0, 35.2, 31.3, 28.0, 20.5, 9.2; MS m/z [M+H]⁺ 622.3.

Intermediate B5 is prepared as follows:

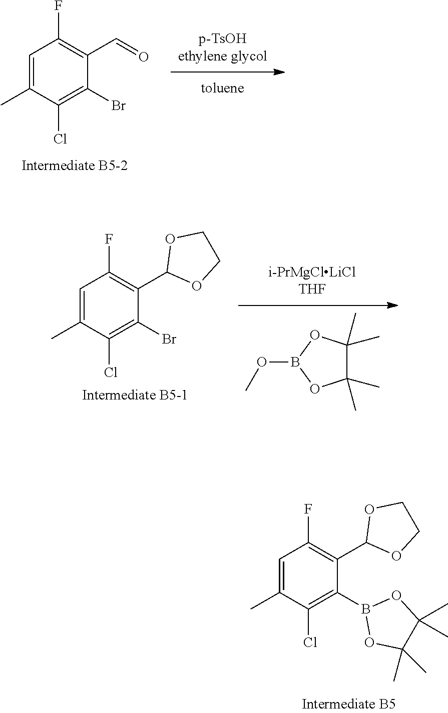

### 2-(2-Bromo-3-chloro-6-fluoro-4-methylphenyl)-1,3-dioxolane (Intermediate B5-1)

To a glass line reactor equipped with an impeller stirrer, a MTBE solution of 2-bromo-3-chloro-6-fluoro-4-methylbenzaldehyde (B5-2, 517.9 Kg, assay: 11.7%, see page 31 of US2019/248767) was added under N₂ and concentrated to 120-180 Kg under vacuum. Toluene (266 Kg) was charged and the mixture was then concentrated to 120-180 Kg. The mixture was cooled to 20-30° C. p-TsOH·H₂O (2.3 Kg), ethylene glycol (25.6 Kg) and toluene (320 Kg) were added sequentially and the resulting solution was heated to 55-62° C. and concentrated to 300-360 Kg at the same temperature. Toluene (320 Kg) was charged and the mixture was concentrated to 300-360 Kg at 55-62° C. The toluene addition/distillation procedure was repeated until the full conversion of B5-2. The reaction mixture was cooled to 20-30° C. and washed with 7% NaHCO₃ aqueous solution (120 Kg) and 5% NaCl aqueous solution (138 Kg). After the phase separation, the organic layer was diluted with THF (210 Kg) and concentrated below 45° C. under vacuum to 120-180 Kg. The THF addition/distillation procedure was repeated until the water content of the solution≤0.2% (KF). The residue was cooled to 20-30° C. to afford a THF/toluene solution of the title compound (268.2 Kg, yield: >99%, purity: 92.2%, assay: 26.8%), which was directly used in the next step without additional purification.

### 2-[2-Chloro-6-(1,3-dioxolan-2-yl)-5-fluoro-3-methylphenyl]-4,4,5,5-tetramethyl-1,3,2-dioxaborolane (Intermediate B5)

To a glass line reactor equipped with an impeller stirrer, a toluene/THF solution of 2-(2-bromo-3-chloro-6-fluoro-4-methylphenyl)-1,3-dioxolane (B5-1, 268.2 Kg, assay: 26.8%) and THF (345 Kg) were added under N₂. The solution was cooled to −10-0° C. followed by the slow addition of i-PrMgCl·LiCl (2.0 M in THF, 182 Kg) below 0° C. The resulting solution was allowed to stir for another 3-5 h, after which, 2-methoxy-4,4,5,5-tetramethyl-1,3,2-dioxaborolane (69 Kg) was slowly added under −10-0° C. The resulting solution was warmed to 20-30° C. and stirred for another 3-5 h. After full conversion, the mixture was diluted with MTBE (497 Kg) and quenched by 10% citric acid aqueous solution (340 Kg) under 0-10° C. The aqueous layer was then extracted with MTBE (178 Kg) and the combined organic layer was sequentially washed with 10% citric acid aqueous solution (354 Kg), 12.5% NaCl aqueous solution (284 Kg×2), water (284 Kg) and concentrated to 140-210 L under vacuum. MTBE (155-200 Kg) and water (50-100 Kg) were charged and the organic layer was concentrated below 45° C. under vacuum to 70-140 Kg. n-Heptane (98 Kg) was charged under 40-50° C. and the resulting mixture was cooled to 0-10° C. and stirred for 2-5 h. B5 seeds (the seeds could be obtained from the same manufacturing batch) was added. The suspension was allowed to stir under 0-10° C. for 2-5 h then filtered. The wet cake was washed with n-heptane (50 Kg) and dried under vacuum to afford the title compound as a white powder (55.35 Kg, yield: 66%). The procedure has been scaled up to 196 Kg. ¹H NMR (400 MHz, DMSO-d₆) δ 7.29 (d, J=12.0 Hz, 1H), 5.97 (s, 1H), 4.03-3.89 (m, 4H), 2.31 (s, 3H), 1.33 (s, 12H); ¹³C NMR (100 MHz, DMSO-d₆) δ 159.5, 157.1, 138.5 (d, J=8.0 Hz), 131.6 (d, J=2.0 Hz), 127.4 (d, J=13.0 Hz), 118.5 (d, J=23.0 Hz), 98.0, 83.7, 64.7, 25.2, 19.7; MS m/z [M+H]⁺ 343.1.

### Step 4. tert-Butyl 6-[(4M)-4-(2-{(E)-[(benzyloxy)imino]methyl}-6-chloro-3-fluoro-5-methylphenyl)-5-methyl-3-(1-methyl-1H-indazol-5-yl)-1H-pyrazol-1-yl]-2-azaspiro[3.3]heptane-2-carboxylate (Intermediate B8)

Step 4a: To a 1 L Radley reactor equipped with an impeller stirrer, tert-butyl 6-[(4M)-4-[2-chloro-6-(1,3-dioxolan-2-yl)-5-fluoro-3-methylphenyl]-5-methyl-3-(1-methyl-1H-indazol-5-yl)-1H-pyrazol-1-yl]-2-azaspiro[3.3]heptane-2-carboxylate (B6, 27.87 g, 44.80 mmol), AcOH (126.62 g) and water (59.28 g) were added. The reaction mixture was stirred under 35° C. for 25 h. Step 4b: After cooling to 0° C., MeOH (260.96 g), NaOAc (4.04 g, 49.28 mmol) and BnONH₂·HCl (7.87 g, 49.28 mmol) were added sequentially and the mixture was stirred under 0° C. for another 5-6 h. B8 crystal seeds (0.025 g; the seeds could be obtained from the same manufacturing batch) was added and the resulting suspension was stirred under 0° C. of 1 h, then the warmed to 25° C. After 1 h stirring under the same temperature, water (164.12 g) was added dropwise in 3 h and the resulting suspension was stirred for 16 h under 25° C. then filtered under vacuum. The wet cake was washed with MeOH/water=2/1 (76.19 g), water (89.20 g×3) and dried under vacuum to afford the title compound as a white powder (24.03 g, yield: 78%, >99.5% e.e.). The procedure has been scaled up to 200 Kg. ¹H NMR (400 MHz, DMSO-d₆) δ 7.95 (s, 1H), 7.68 (s, 1H), 7.57 (s, 1H), 7.50 (d, J=8.8 Hz, 1H), 7.44 (d, J=11.0 Hz, 1H), 7.32 (dd, J₁=8.8 Hz, J₂=1.4 Hz, 1H), 7.24 (dt, J₁=6.0 Hz, J₂=3.0 Hz, 3H), 7.16 (dd, J₁=6.7 Hz, J₂=2.8 Hz, 2H), 4.96-4.87 (m, 2H), 4.82 (p, J=7.9 Hz, 1H), 3.99 (s, 5H), 3.91 (s, 2H), 2.87-2.74 (m, 2H), 2.74-2.64 (m, 2H), 2.40 (s, 3H), 1.93 (s, 3H), 1.38 (s, 9H); ¹³C NMR (100 MHz, DMSO-d₆) 5159.10, 156.57, 155.36, 147.13, 143.56 (d, J=3.0 Hz), 139.64 (d, J=10.1 Hz), 138.89, 137.86, 137.20, 134.78 (d, J=4.0 Hz), 132.69, 130.95 (d, J=3.0 Hz), 128.06 (d, J=14.1 Hz), 127.71, 125.94, 124.81, 123.46, 118.94 (d, J=12.1 Hz), 118.40, 118.17, 117.74, 112.16, 109.53, 78.39, 75.43, 47.03, 39.62, 35.27, 31.29, 28.02, 20.65, 9.26; MS m/z [M+H]⁺ 683.3.

### Step 5.

### Experimental Procedure 1:

### tert-Butyl 6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptane-2-carboxylate-propan-1-ol (1/x) (Intermediate B10)

To a 500 mL Radley reactor equipped with an impeller stirrer, tert-Butyl 6-[(4M)-4-(2-{(E)-[(benzyloxy)imino]methyl}-6-chloro-3-fluoro-5-methylphenyl)-5-methyl-3-(1-methyl-1H-indazol-5-yl)-1H-pyrazol-1-yl]-2-azaspiro[3.3]heptane-2-carboxylate (B8, 150 g, 219.56 mmol), sodium acetate (27.0 g, 329.34 mmol), NMP (291.3 g) and hydrazine monohydrate (purity: 98-100%, 87.9 g, 1756.48 mmol) were added sequentially under N₂. The resulting suspension was heated to 80° C. in 1 h and stirred for 48 h. After the full conversion, the mixture was cooled to 20° C., then diluted with ethyl acetate (750 ml) and 10% NaCl aqueous solution (750 g). The mixture was cooled to 10° C. and stirred for 30 min. The organic layer was separated and washed with a citric acid/NaCl solution (prepared by adding 31.2 g of citric acid to 1 L of 10% NaCl aqueous solution) for 3 times (750 g*3) and 2% NaHCO₃ aqueous solution (750 g) once. The pre-treated resin (from 30.0 g of the Amberchrom 50WX4, a 4% cross-linked styrene-divinylbenzene based cation exchange resin with sulfonic acid functional groups) was added and the resulting suspension was stirred at 25° C. for 8 h followed by the filtration through a pad of cellflock (powdered cellulose). The cake was washed with ethyl acetate (300 mL×3) and the combined filtrate was concentrated to ca. 835 mL. The distillation residue is heated to 50° C. 1-Propanol (193 g) and B10 crystal seeds (the seeds could be obtained from the same manufacturing batch) were added sequentially and the resulting suspension was stirred at 50° C. for 1 h. Extra 1-propanol (64 g) was added and the suspension was stirred at 50° C. for 3 h, followed by the slow addition of n-heptane (2.31 Kg) in 6 h. The suspension was then cooled to 5° C. in 6 h, held for an extra hour, then filtered under vacuum. The wet cake was washed with ethyl acetate:n-heptane=1:3 (228 g) twice and dried under vacuum to afford the title compound as a white powder (115.50 g, yield: 83%, >99.0% e.e.). The procedure has been scaled up to 200 Kg. ¹H NMR (400 MHz, DMSO-d₆) δ 13.07 (s, 1H), 7.87 (s, 1H), 7.57 (s, 1H), 7.54 (s, 1H), 7.44-7.36 (m, 2H), 7.31-7.26 (m, 1H), 4.86 (p, J=8.0 Hz, 1H), 4.02 (s, 2H), 3.94 (s, 5H), 2.93-2.70 (m, 4H), 2.48 (s, 3H), 2.02 (s, 3H), 1.40 (s, 9H); ¹³C NMR (100 MHz, DMSO-d₆) δ 155.4, 147.4, 138.8, 138.2, 137.6, 134.1, 133.0, 132.5, 126.8, 126.5, 125.1, 124.9, 123.4, 123.3, 117.7, 112.8, 110.9, 109.2, 78.4, 47.0, 35.2, 31.3, 28.0, 21.5, 9.7; MS m/z [M+H]⁺ 572.3.

Pre-treatment of impurity scavenger Amberchrom 50WX4 (200-400 mesh): Amberchrom 50WX4 (30 g) was suspended in methanol (80 g) and the resulting mixture was stirred at room temperature for 10 min then filtered. The wet cake was rinsed with ethyl acetate (75 g×2) and dried under reduced pressure.

### Experimental Procedure 2:

### tert-Butyl 6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptane-2-carboxylate (amorphous) (Intermediate B10)

To a 100 mL three-necked flask equipped with a magnetic stirring bar, tert-butyl 6-[(4M)-4-(2-{(E)-[(benzyloxy)imino]methyl}-6-chloro-3-fluoro-5-methylphenyl)-5-methyl-3-(1 methyl-1H-indazol-5-yl)-1H-pyrazol-1-yl]-2-azaspiro[3.3]heptane-2-carboxylate (B8, 15 g, 21.52 mmol), sodium acetate (0.88 g, 10.76 mmol), NMP (77.3 g), hydrazine monohydrate (85 wt. %, 10.3 g, 172.12 mmol) and water (3.1 g) were added sequentially under N₂. The resulting mixture was heated to 80° C. in 1 h and stirred for 50 h. After the full conversion, the reaction mixture was cooled to 40° C. and added dropwise to H₂O (250 mL) in 1 h. The resulting suspension was stirred at 40° C. for 1 h, cooled to 10° C. and stirred for another hour. After filtration, the wet cake was washed with water (45 mL×2) and re-dissolved in DCM (120 mL). 2% NaHCO₃ aqueous solution (80 g) was added and the mixture was stirred at 25° C. for 15 min. The organic layer was removed and the aqueous layer was then washed with DCM (30 mL). The combined organic layer was treated with Amberchrom 50WX4 (200-400 mesh, 5 g) at 25° C. for 16 h and filtered. The filtrate was concentrated under vacuum to about 60 g. Ethyl acetate (200 g) was added and the resulting solution was concentrated to about 90 g. The above procedure was repeated twice to replace DCM with ethyl acetate. The residue (˜85 g) was heated to 50° C. followed by the dropwise addition of n-heptane (115 mL) in 30 min. B10 crystal seeds (the seeds could be obtained from the same manufacturing batch) was added and the mixture was then stirred at 50° C. for 6 h. The rest portion of n-heptane (80 mL) was added in 2 h and the suspension was stirred at 50° C. for 3 h, cooled to 20° C. in 3 h and stirred for another 3 h. After filtration, the wet cake was washed with ethyl acetate:n-heptane=1:3 (30 g×3) and dried under vacuum to afford the title compound as a white powder (12.31 g, yield: 76%, 96.4% e.e.). The procedure has been scaled up to 3.6 Kg.

### Step 6. 6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-ium hydrogen sulfate (IUPAC name) (Intermediate B11)

To a glass line reactor equipped with an impeller stirrer was added 45% H₂SO₄ aqueous solution (264 Kg) under N₂. tert-butyl 6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptane-2-carboxylate-propan-1-ol (1/x) (B10, 35 Kg) was dissolved in THE (52 Kg) and water (2.8 Kg) and the resulting solution was added slowly to the reactor in 1 h under 20° C. After the addition finished, the funnel was rinsed with THE (5 Kg) and the reaction mixture was stirred under 20° C. for another 60 min. After the full conversion, the mixture was cooled to 10° C. 25% Ammonium hydroxide aqueous solution (97.9 Kg) was slowly added in 30 min followed by the addition of isopropanol (77.2 Kg). Another portion of 25% ammonium hydroxide aqueous solution (74.0 Kg) was added to adjust the pH range to 7-7.5. The aqueous layer was removed and acetonitrile (166 Kg) was slowly added to the organic layer in 1.5 h under 25° C. B11 Seeds (from preceding reaction without seeds) was added and the resulting suspension stirred for 3 h. Another portion of acetonitrile (95 Kg) was slowly added in 6 h. The resulting suspension was cooled to −15° C. in 3 h and aged for another 8 h. After the filtration under −15° C., the wet cake was washed with acetonitrile (75 Kg×2) and partially dried under vacuum at 10° C. for 24 h to afford the title compound as a white solid which was stored at −15° C. under argon and directly used in the next step without additional purification (32 Kg, yield: 92%). MS m/z [M+H]⁺ 472.2.

### Step 7. 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one-propan-2-ol (1/1)

To a glass line reactor equipped with an impeller stirrer, 6-[(4M)-4-(5-chloro-6-methyl-1H-indazol-4-yl)-5-methyl-3-(1-methyl-1H-indazol-5-yl)-1H-pyrazol-1-yl]-2-azaspiro[3.3]heptan-2-ium hydrogen sulfate (B11 wet cake, 3.83 Kg), IPA (9.06 Kg) and water (6.01 Kg) were added under N₂. The resulting clear solution was stirred at 15° C. for 20 min then cooled to 0-5° C. 5% NaHCO₃ aqueous solution (8.21 Kg) was added in 0.5-1 h to maintain the internal temperature less than 10° C. Additional NaHCO₃ powder (0.61 Kg) was added in one portion followed by the dropwise addition of acrylic anhydride (0.25 Kg) in 15 min. The dropping funnel was rinsed with DCM (1.0 Kg) and the mixture was stirred under 0-5° C. for 1.5 h. After the full conversion, the mixture was warmed to 20-30° C. and stirred for 30 min. DCM (18.10 Kg) was added and the organic layer was separated and washed with 2% NaCl aqueous solution (11.73 Kg). The organic layer was concentrated while extra IPA (7.5 Kg) was added in portions to fully replaced DCM with IPA and to control the water content<0.5%. After the distillation, the residue was cooled to 30° C. IPA (14 Kg) was added and the diluted mixture was cooled to 20° C. and stirred for 24 h. The resulting suspension was filtered under vacuum and the wet cake was washed with IPA (1.8 Kg) then dissolved in DCM (15 Kg). Silica gel (0.6 Kg) was added and the suspension was allowed to stir under 25° C. for another 2.5 h. After the filtration, the DCM in the filtrate was again fully replaced with IPA under vacuum and the resulting IPA suspension was cooled to 20° C. in 2 h and aged for another 18 h. After filtration, the wet cake was rinsed with IPA (2.2 Kg) and dried under vacuum to afford the tile compound as a white powder (0.69 Kg, yield: 60%, 99.6% e.e.). The procedure has been scaled up to 77 Kg. ¹H NMR (400 MHz, DMSO-d₆) δ 13.10 (s, 1H), 7.88 (s, 1H), 7.58 (s, 1H), 7.54 (s, 1H), 7.43-7.36 (m, 2H), 7.30 (d, J=8.0 Hz, 1H), 6.38-6.25 (m, 1H), 6.11 (dt, J₁=16.0 Hz, J₂=4.0 Hz, 1H), 5.72-5.63 (m, 1H), 4.96-4.84 (m, 1H), 4.39 (s, 1H), 4.35-4.27 (m, 2H), 4.11 (s, 1H), 4.04 (s, 1H), 3.94 (s, 3H), 3.83-3.72 (m, 1H), 2.97-2.73 (m, 4H), 2.48 (s, 3H), 2.03 (s, 3H), 1.05 (s, 3H), 1.03 (s, 3H); ¹³C NMR (100 MHz, DMSO-d₆) δ 164.4, 147.4, 138.8, 138.3, 137.8, 137.7, 134.1, 133.0, 132.5, 127.0, 126.8, 126.5, 126.3, 125.1, 124.9, 123.4, 123.3, 117.8, 112.8, 110.9, 109.2, 62.0, 61.9, 60.6, 59.7, 58.4, 47.0, 46.9, 35.2, 31.5, 31.4, 25.4, 21.6, 9.7; MS m/z [M+H]⁺ 526.2.

This isopropyl solvate form of Compound A can be transformed into another solvate or the hydrate as is described in WO 2021/1224222 A1 or in the following:

### Example 2

A compound of formula (Ia) as 2-propanol form obtained according to Step 7 of Example 1 was dried at ambient conditions overnight which provided Compound A in the crystalline hydrate (Modification HA) form and showed the XRPD characteristics shown above or in WO 2021/124222 A1.

### Example 3: Alternative Preparation of Crystalline Hydrate (Modification HA) Preparation

25 mg of a compound of formula (Ia) (Example 1 Step 7) was added to 0.1 mL of methanol. The resulting clear solution was stirred at 25° C. for 3 days. Crystalline hydrate (Modification HA) obtained in example 1A was added as seeds to the resulting solution. The resulting suspension was equilibrated for another 1 day, after which a solid precipitated out. The solid was collected by centrifuge filtration and dried at ambient condition overnight. After drying at ambient condition overnight, the wet cake produced crystalline hydrate (Modification HA).

1. A process for preparing a compound of formula (I), or a salt, or hydrate or a solvate thereof, 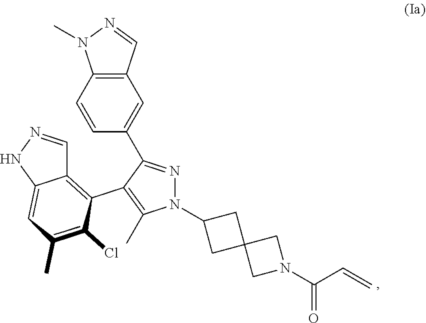
comprising reacting Compound B11b*, or a salt thereof, 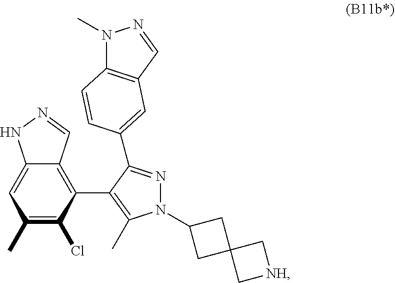
with acrylic acid or a reactive derivative of acrylic acid to form a compound of formula (Ia) and optionally subsequently forming a salt or hydrate or solvate of the compound of formula (Ia).

2. A process for preparing a compound of formula (Ia), or salt, or a hydrate or a solvate thereof, 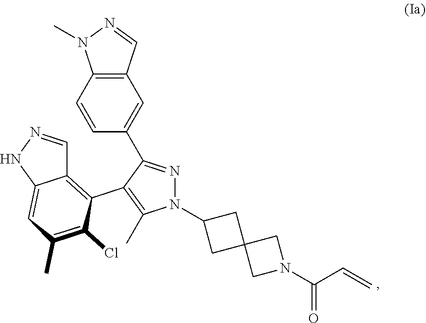
comprising an acylation of the compound Intermediate B11*, 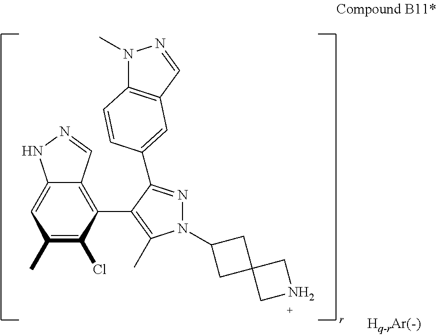
wherein r is 1, 2, 3, 4, or 5, especially 1 to 3, e.g. 1 or 2, q is 1, 2, 3, 4 or 5, especially 1 to 3, e.g. 1 or 2, and A is an acid anion of an organic or an inorganic acid, or the deprotonated free form thereof without the acid anion,
with acrylic acid or a reactive derivative thereof;
and, optionally, forming the salt of a compound of formula (Ia) or converting a hydrate or solvate of Compound of formula (Ia) into the free form or a different hydrate or solvent, or converting the free form of Compound of formula (Ia) into a hydrate or solvate thereof.

3. The process of claim 1, or a process of preparing Intermediate B11*, comprising deprotecting Intermediate B10*, 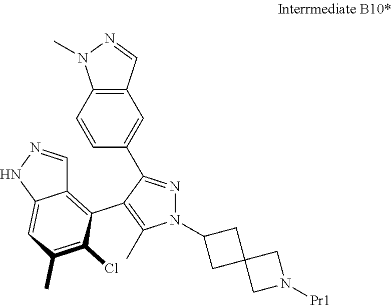
wherein Pr1 is a protecting group, to form Compound B11*.

4. The process of any one of the previous claims or a process for preparing Intermediate B10*, comprising reacting hydrazine, or a hydrate or solvate thereof, with Intermediate B8*, 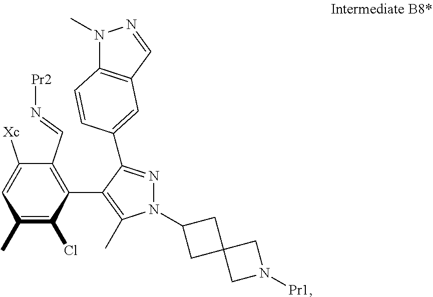
wherein Pr1 is a nitrogen-protecting group, Pr2 is a protected hydroxyl group or an unsubstituted or substituted amino group, and Xc is halogeno or pseudohalogeno.

5. The process of any one of the previous claims or a process for preparing Intermediate B8*, comprising reacting Intermediate B7*, 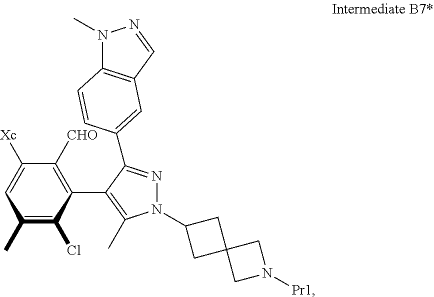
wherein Pr1 is a nitrogen-protecting group and Xc is halogeno or pseudohalogeno, with a hydroxyolamine derivative of the formula Pr2-NH₂, wherein Pr2 is a protected hydroxyl group or an unsubstituted or substituted amino group, with a hydroxylamine derivative of the Formula Pr2-NH₂, wherein Pr2 is a protected hydroxyl group or an unsubstituted or substituted amino group.

6. The process of any one of the previous claims, or a process for preparing Intermediate B7*, comprising converting the protected formyl group Q of Intermediate B6* to form Intermediate B7*, 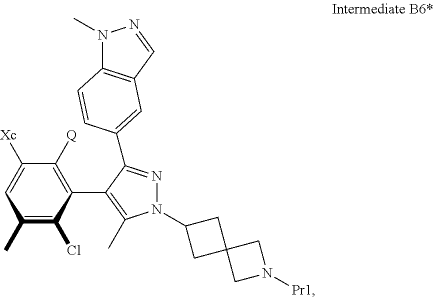
wherein Q is a formyl group in the form of an acetal or of a Schiff base, Pr1 is a nitrogen-protecting group and Xc is halogeno or pseudohalogeno, optionally in the presence of an acid.

7. A process for preparing Intermediate B8* according to any one of the previous claims wherein the process steps are conducted in a one-pot synthesis.

8. A process according to any one of the previous claims or a process for preparing Intermediate B6*, comprising coupling Intermediate B4*, 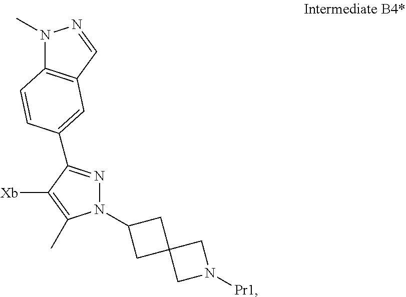
wherein Pr1 is a nitrogen-protecting group and
(a) Xb is hydrogen, halogeno, —OSO₂RA in which RA is C₁-C₄-alkyl, fluoro-C₁-C₆-alkyl or substituted phenyl, or ORB wherein RB is C₁-C₄-alkyl, if Lb is borono (—B(OH)₂), a boronic diester moiety, BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li;
or (b) Xb is borono (—B(OH)₂), a boronic diester moiety, BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li; if Lb is hydrogen, halogeno, —OSO₂RA in which RA is C₁-C₄-alkyl, fluoro-C₁-C₆-alkyl or substituted phenyl, or ORB wherein RB is C₁-C₄-alkyl,
in the presence of a chiral (enantiomerically pure) mono- or (especially) bisphosphine ligand catalyst and a palladium source reagent with an Intermediate B5*, 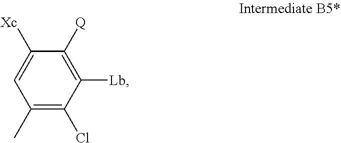
wherein Q is formyl or in particular a formyl group in the form of an acetal or a Schiff base,
Lb is (a′) (in particular) borono (—B(OH)₂), a boronic diester moiety, BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li, if Xb is as just defined above for Xb under (a),
or (b′) Lb is hydrogen, halogeno, —OSO₂RA in which RA is C₁-C₄-alkyl, fluoro-C₁-C₆-alkyl or substituted phenyl, or ORB wherein RB is C₁-C₄-alkyl, if Xb is as just defined under (b) for Xb above;
and
Xc is halogeno or pseudohalogeno, to form Intermediate B6*.

9. The process of any one of the preceding claims, or a process for preparing Intermediate B4*, wherein (a) Xb is halogeno, —OSO₂RA in which RA is C₁-C₄-alkyl, fluoro-C₁-C₆-alkyl or substituted phenyl, or ORB wherein RB is C₁-C₄-alkyl, if Lb is borono (—B(OH)₂), a boronic diester moiety, BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li;
or (b) Xb is borono (—B(OH)₂), a boronic diester moiety, BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li; if Lb is hydrogen, halogeno, —OSO₂RA in which RA is C₁-C₄-alkyl, fluoro-C₁-C₆-alkyl or substituted phenyl, or ORB wherein RB is C₁-C₄-alkyl,
comprising reacting an Intermediate B3*, 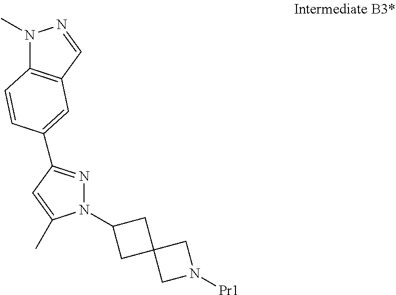
wherein Pr1 is a nitrogen-protecting group, with a reagent capable of inserting the group Xb wherein Xb is Xb is halogeno, —OSO₂RA in which RA is C₁-C₄-alkyl, fluoro-C₁-C₆-alkyl or substituted phenyl, or ORB wherein RB is C₁-C₄-alkyl, if Lb is borono (—B(OH)₂), a boronic diester moiety, BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li;
or (b) Xb is borono (—B(OH)₂), a boronic diester moiety, BF₃K, MgX, in which X is Cl, Br or I, Zn(RZ), in which RZ is halogen or alkyl; Sn(RD)(RE)(RF), in which each of RD, RE, RF is methyl, n-butyl; Si(RG)(RH)(RI), in which each of RG, RH, RI is hydrogen, fluoro, hydroxy, alkyl, alkoxy, or Li; if Lb is hydrogen, halogeno, —OSO₂RA in which RA is C₁-C₄-alkyl, fluoro-C₁-C₆-alkyl or substituted phenyl, or ORB wherein RB is C₁-C₄-alkyl.

10. The process of any one of the preceding claims, or a process for preparing Intermediate B3*, comprising reacting an Intermediate B2*, 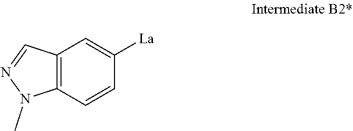
wherein La is borono, a boronic diester moiety, BF₃K, MgX, in which X is Cl, Br or I, Zn(RZ), in which RZ is halogen or alkyl, Sn(RD)(RE)(RF), in which each of RD, RE, RF is methyl, n-butyl, Si(RG)(RX)(RY), in which each of RG, RX, RY is hydrogen, fluoro, hydroxy, alkyl, alkoxy, or Li, in the presence of a cross-coupling catalyst with Intermediate B1*, 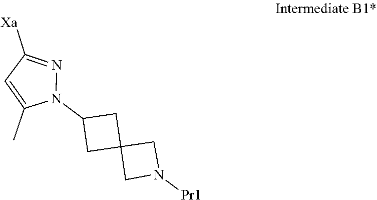
wherein Pr1 is a nitrogen-protecting group and Xa is hydrogen, halogeno, —OSO₂RA in which RA is C₁-C₄-alkyl, fluoro-C₁-C₆-alkyl or substituted phenyl, or ORB wherein RB is C₁-C₄-alkyl, to yield Intermediate B3*.

11. The process of any one of the preceding claims, wherein in the Intermediate B4*
Pr1 is a nitrogen-protecting group selected from the group consisting of tert-butyoxycarbonyl, carbobenzoxycarbonyl (Cbz), benzyl (Bn), methoxybenzyl (MPM), trifluoroacetyl, acetyl, fluoren-9-yl-methoxycarbonyl (Fmoc) and trityl (Tr);
and Xb is hydrogen, halogeno, a mesylate moiety, a triflate moiety or a tosylate moiety;
and in the Intermediate B5*, Q is selected from the group consisting of 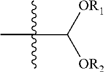
in which R₁ and R₂ are independently selected from alkyl or arylalkyl or together form an alkenyl bridge that may be unsubstituted or substituted with one or more moieties selected from alkyl, aryl and arylalkyl; some preferred moities are 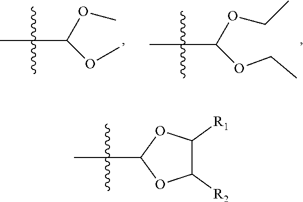
in which R₁ and R₂ are independently selected from hydrogen and alkyl and further aryl, 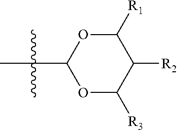
in which R₁ and R₂ are independently selected from hydrogen, alkyl and further aryl;
the following groups are special examples: 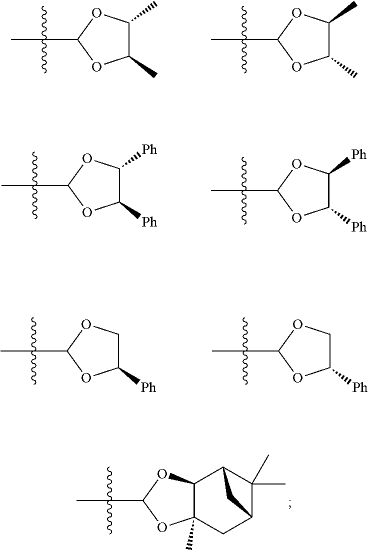
and further
—CH═N—R′ in which R′ is selected from the group consisting of alkyl, aryl, alkoxy and S(═O)—R″ wherein R″ is selected from alkyl, aryl and alkoxy, such as —S(═O)-alkyl (e.g. tert-butyl) or —S(═O)phenyl;
the chiral (enantiomerically pure) mono- or (especially) bisphosphine ligand catalyst is selected from the group consisting of 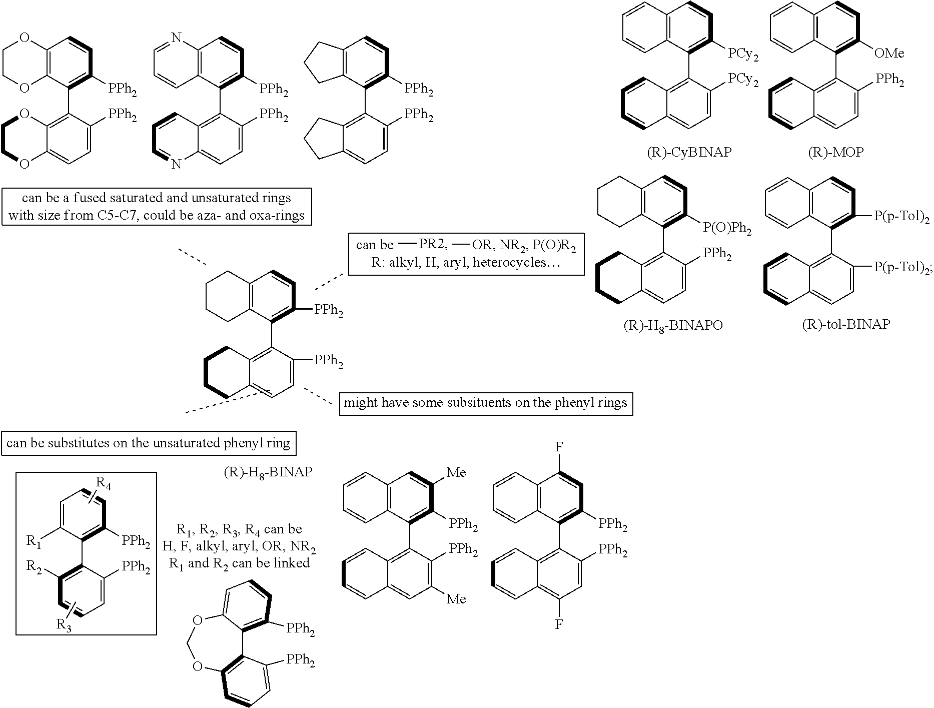
and especially from the
Group 1 consisting of
Group 1: 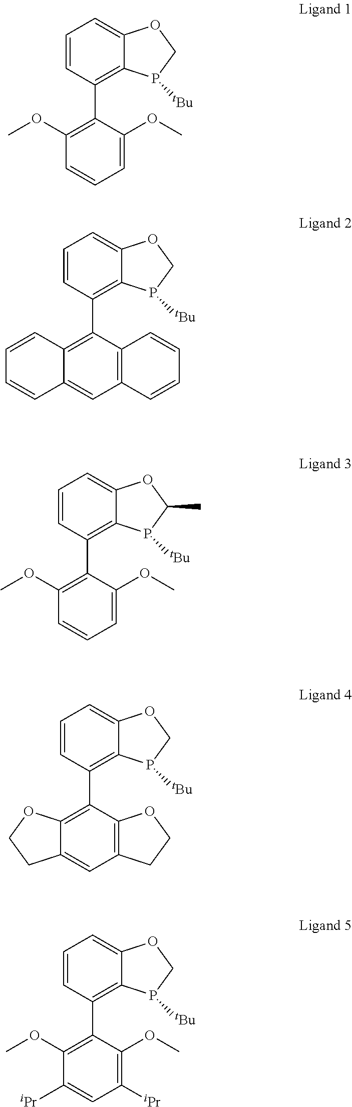 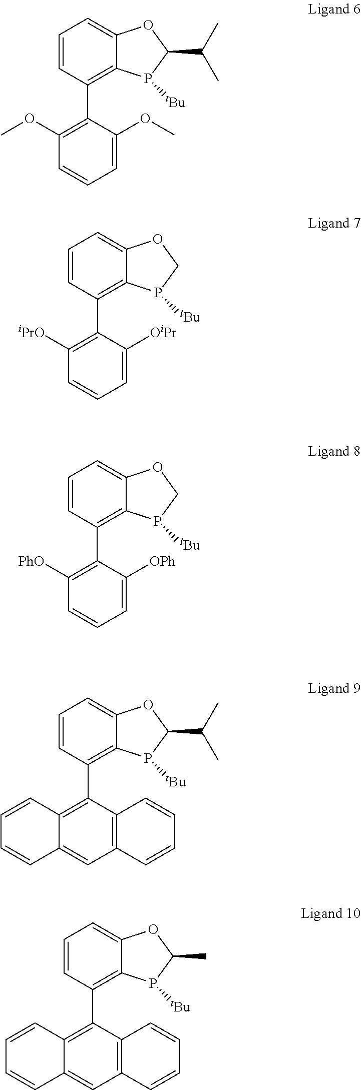 [SMILES: COc1cccc(OC)c1-c1cccc2c1[P@@](C(C)(C)C)[C@H](C(C)C)O2 ; CC(C)Oc1cccc(OC(C)C)c1-c1cccc2c1[P@@](C(C)(C)C)CO2 ; CC(C)(C)[P@@]1COc2cccc(-c3c(Oc4ccccc4)cccc3Oc3ccccc3)c21 ; CC(C)[C@@H]1Oc2cccc(-c3c4ccccc4cc4ccccc34)c2[P@@]1C(C)(C)C ; C[C@@H]1Oc2cccc(-c3c4ccccc4cc4ccccc34)c2[P@@]1C(C)(C)C] 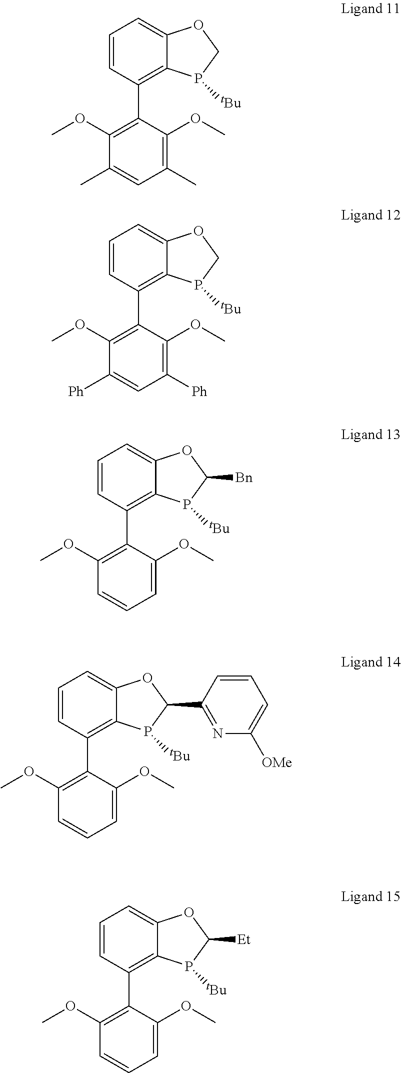 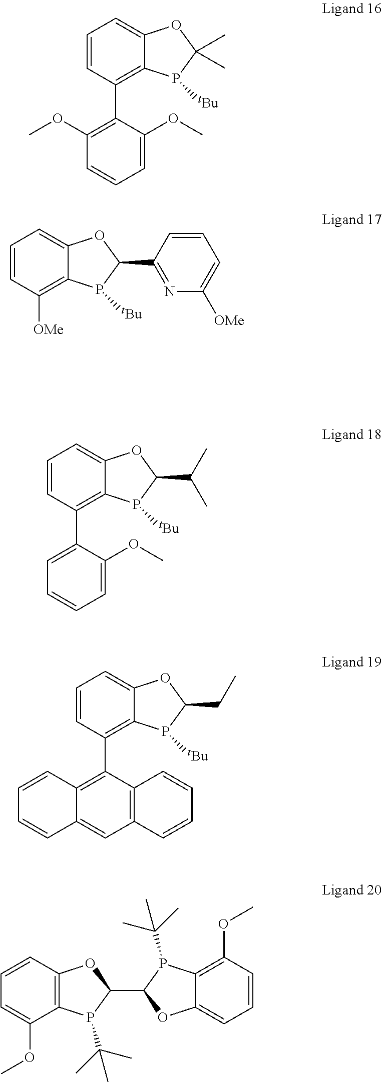
from
the Group 2 consisting of:
Group 2: BINAP and related ligands: (most important are Ligand 21, 22, 23 or 24) 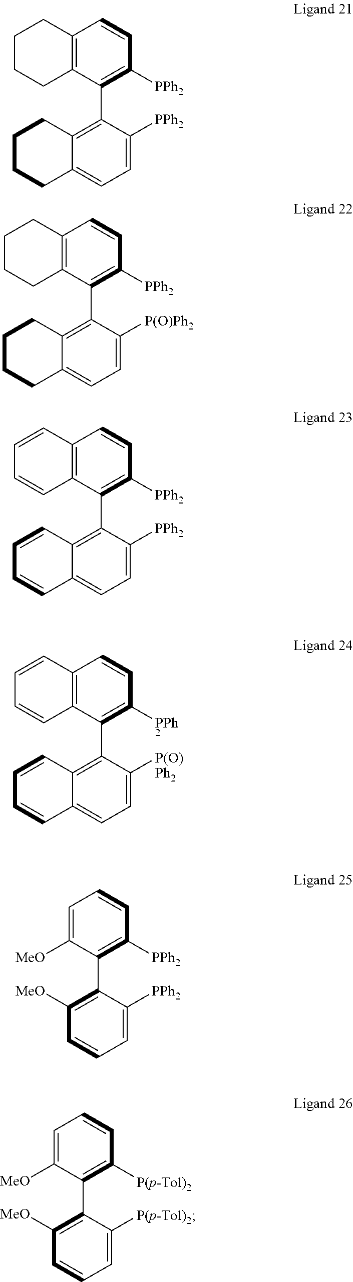
and
from the Group 3 consisting of:
Group 3: Other ligands: 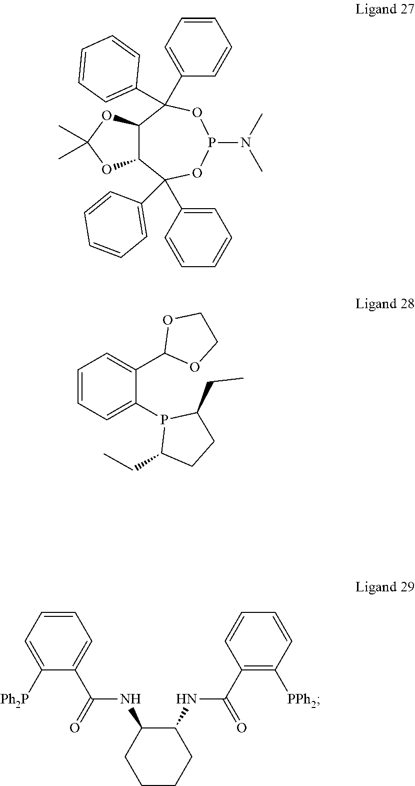
the palladium source reagent is selected from the group consisting of palladium source, any Pd salt or complex can be employed, such as Pd(OAc)₂, Pd(OPiv)₂, Pd(OCOEt)₂, PdCl₂, PdBr₂, PdI₂, Pd(OH)₂, PdSO₄, Pd(TFA)₂, Pd(dba)₂, Pd₂(dba)₃, Pd(acac)₂, Pd₂(dba)₃·CHCl₃, Pd(PPh₃)₄, Pd(PPh₃)₂Cl₂, Pd(CH₃CN)₂Cl₂, Pd(PhCN)₂Cl₂, [Pd(π-allyl)Cl]₂, [Pd(π-cinnamyl)Cl]₂, Pd[P(o-Tol)₃]₂, Pd/C, Pd(OH)₂/C, [Pd-G1]₂, [Pd-G2]₂, [Pd-G3]₂, [Pd-G4]₂, Pd(BINAP)Cl₂, Pd(dppe)Cl₂, Pd(dppp)Cl₂, Pd(dppb)Cl₂, and Pd(dppf)Cl₂; Pd(II) salts or complexes selected, e.g. for bisphosphone ligands Pd(II), for example selected from Pd(OAc)₂, Pd(TFA)₂ and Pd(PhCN)₂Cl₂; or for bisphosphine monoxide ligands Pd(0) can be used, e.g. Pd₂(dba)₃ or Pd(dba)₂;
where the reaction is preferably conducted in the presence of a base, such as selected from the group consisting of Cs₂CO₃, CsOH·H₂O, CsHCO₃, CsOPiv, CsF, CsOAc, K₃PO₄, K₂CO₃, KHCO₃, KF, TMSOK, NaOH, NaHCO₃, KOH, NaOMe, KOMe, NaOEt, KOEt, KO^{t}Bu, NaO^{t}Bu, NaOAc, KOAc, DABCO, DBU, TEA, DIPEA, Cy₂NMe, pyridine, 2,6-lutidine, or the like. Cs₂CO₃, especially in micronized form, is a preferred example; in one or more solvents selected from the group consisting of DMF, DMSO, NMP, water, MeOH, EtOH, i-PrOH, tert-amyl alcohol, toluene, o-xylene, m-xylene, p-xylene, 1,3,5-trimethylbenzene, 1,3,5-trifluorobenzene, chlorobenzene, trifluoromethylbenzene, 1,2-difluorobenzene, n-heptane, n-hexane, c-hexane, n-pentane, THF, 2-MeTHF, 1,4-dioxane, MTBE, CPME, i-Pr₂O, n-Bu₂O, Ph₂O, DME (1,2-dimethoxyethane), MeO(CH₂CH₂O)₂Me, cyclohexane, acetonitrile, DCM, Et₃N, DIPEA, 2,6-lutidine, ethyl acetate, i-propyl acetate, t-butyl acetate, MIBK and sulfolane; can, for example, be used; where the sum of water in the reaction mixture preferably is kept at about 1.5 (e.g. 1.2 to 1.8) equivalents per mol of Intermediate B4*; where the reaction preferably takes place at temperatures in the range from 0° C. to the boiling temperature of the reaction mixture, especially at an elevated temperature in the range from 25 to 90° C., such as in the range from 50 to 75° C., e.g. at 60 to 70° C.

12. The process of preparing a compound of formula (Ia), or a salt, or a hydrate or a solvate thereof, comprising the following reactions: 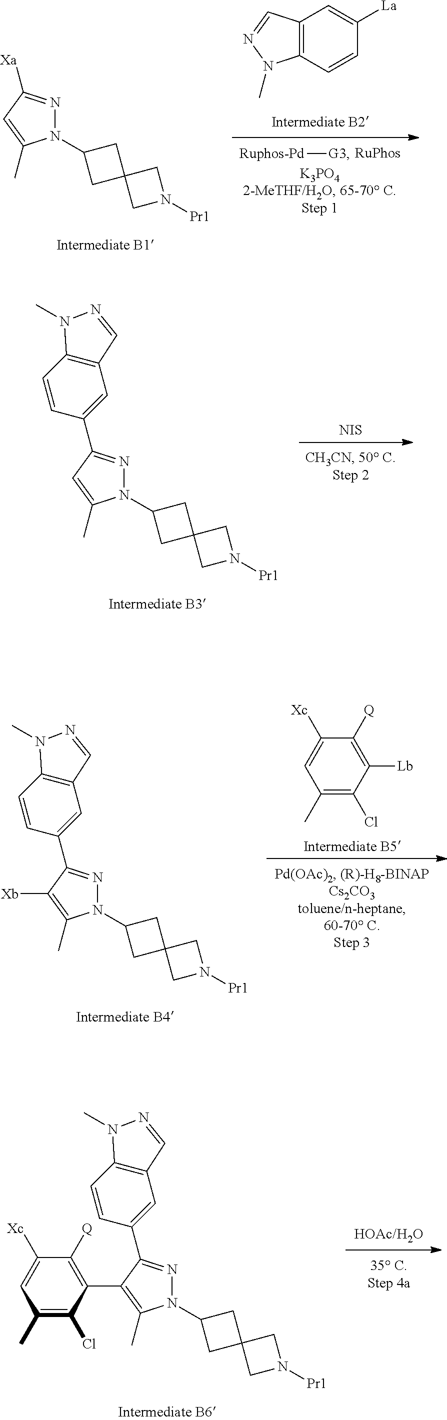 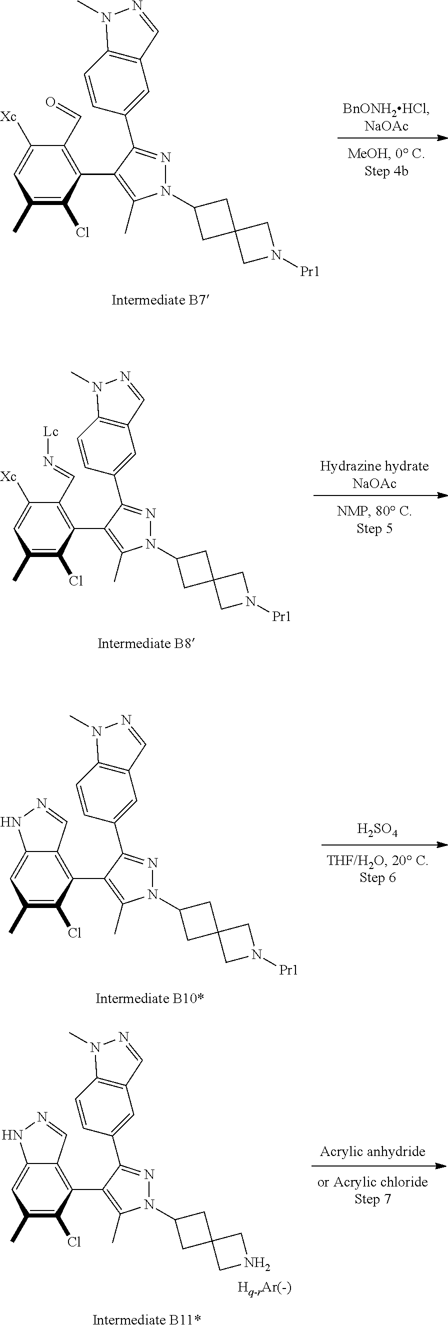 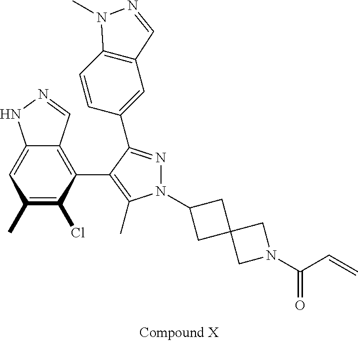
wherein
Xa is iodo or chloro or especially bromo;
Pr1 is tert-butoxycarbonyl
La is —B(OH)₂;
Xb is chloro, bromo or iodo;
Xc chloro or especially fluoro;
Q is a group of the formula 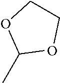
Lb is a group of the formula 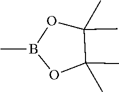
Lc is benzyloxy;
q is 1, 2 or 3;
r is 1, 2 or 3; and
A is Cl⁻, Br⁻, F⁻, HSO₄⁻ or SO₄²⁻.

13. A process for preparing Intermediate B6* comprising coupling Intermediate B4* with Intermediate B5* using a chiral catalyst

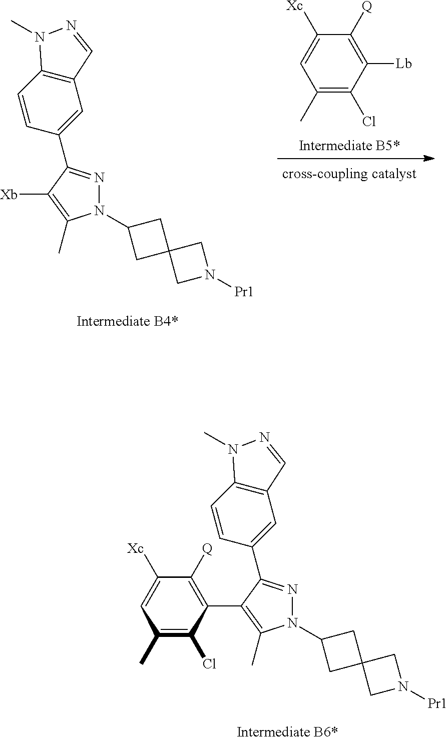

14. A process for preparing a compound of formula (Ia) according to the Scheme below

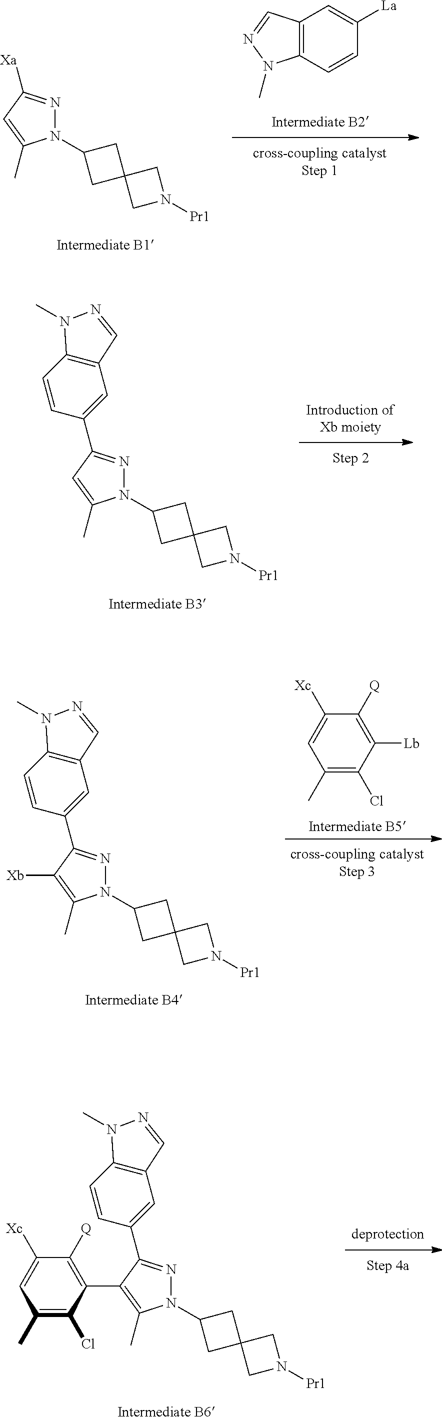

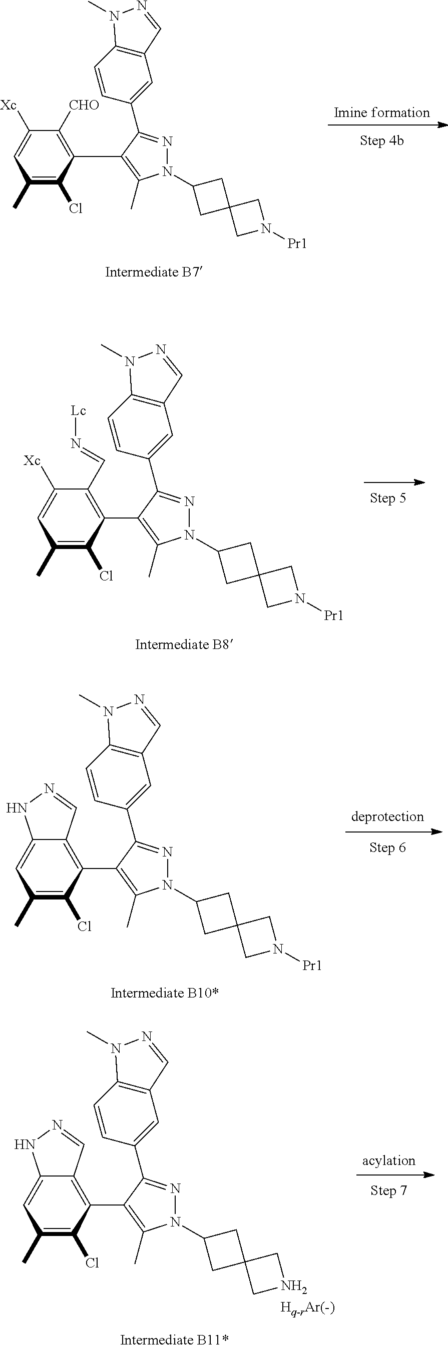

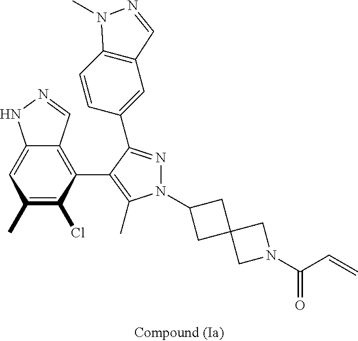

15. Intermediate B6* of the formula 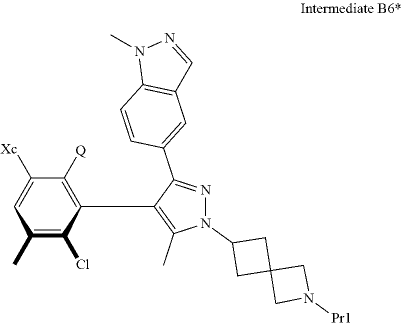
wherein Q is formyl or a formyl group in the form of an acetal or (further) of a Schiff base;
Pr1 is a nitrogen-protecting group; and
Xc is halogeno or pseudohalogeno.

16. Intermediate B7* of the formula 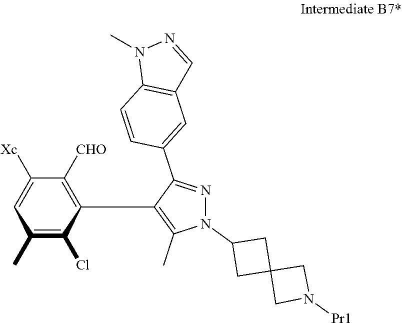
wherein Pr1 is a nitrogen-protecting group and
Xc is halogeno or pseudohalogeno.

17. Intermediate B8* of the formula 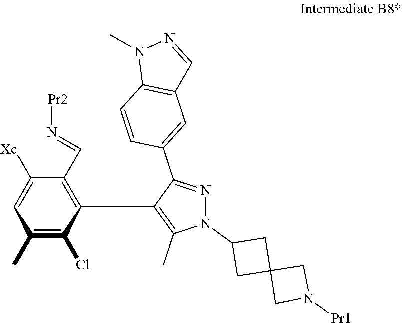
wherein Pr1 is a nitrogen-protecting group,
Pr2 is a protected hydroxyl group or an unsubstituted or substituted amino group,
and Xc is halogeno or pseudohalogeno.

18. Intermediate B10* of the formula 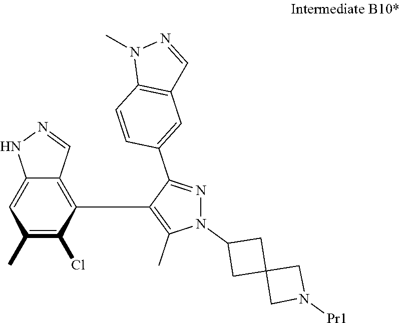
wherein Pr1 is a nitrogen-protecting group.

19. Intermediate B11* of the formula 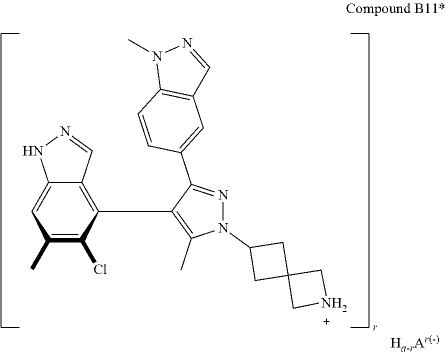
wherein r is 1, 2, 3, 4, or 5, especially 1 to 3, e.g. 1 or 2, q is 1, 2, 3, 4 or 5, especially 1 to 3, e.g. 1 or 2, and A is an acid anion of an organic or an inorganic acid, with acrylic acid or a reactive derivative thereof.

20. A process according to any one of claims 1 to 14, wherein the process is carried out on an industrial scale, e.g. on a greater than 10 g scale or a kilogramme scale.
